(19) United States
(12) Patent Application Publication
(10) Pub. No.: US 2010/0310661 A1
(43) Pub. Date: Dec. 9, 2010

(54) ORAL FORMULATIONS FOR PICOPLATIN

(75) Inventors: Andrew Xian Chen, San Diego, CA (US); Cheni Kwok, San Mateo, CA (US); Christopher A. Procyshyn, Surrey (CA)

Correspondence Address: SCHWEGMAN, LUNDBERG & WOESSNER, P.A., P.O. BOX 2938, MINNEAPOLIS, MN, 55402, US

(73) Assignee: Poniard Pharmaceuticals, Inc., Seattle, WA (US)

(21) Appl. No.: 12/669,274

(22) PCT Filed: Jul. 16, 2008
(86) PCT No.: PCT/US08/08669

§ 371 Date: Jun. 21, 2010

Related U.S. Application Data
(60) Provisional application No. 60/950,033, filed on Jul. 16, 2007.
(60) Provisional application No. 61/043,962, filed on Apr. 10, 2008.

(51) Int. Cl. A61K 31/555; A61K 9/14; A61K 31/704; A61P 35/00

(52) U.S. Cl. 424/489; 514/188; 514/34

(57) ABSTRACT

The invention provides formulations for the organoplatinum anticancer drug picoplatin. Self emulsifying compositions, stabilized nanoparticulate compositions, solid dispersions, and nanoparticulate suspensions in oils are provided, along with methods for preparation of the formulations. The formulations can provide improved oral availability of picoplatin relative a to a simple solution of picoplatin such as in water or normal saline solution and can be used in combination therapy.

### CROSS REFERENCE TO RELATED APPLICATIONS

This application claims priority to U.S. Provisional Application Ser. Nos. 60/950,033, filed Jul. 16, 2007, and 61/043,962 filed Apr. 10, 2008, both entitled “Oral Formulations for Picoplatin”, both of which are incorporated by reference in their entireties herein.

### BACKGROUND

Picoplatin is a new-generation organoplatinum drug that has promise for treatment of various types of malignancies, including those that have developed resistance to earlier organoplatinum drugs such as cisplatin and carboplatin. Picoplatin has shown promise in the treatment of various kinds of cancer or tumor, including small cell lung cancer, colorectal cancer, and hormone-refractory prostate cancer.

Structurally, picoplatin is:

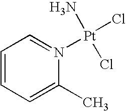

and is named cis-amminedichloro(2-methylpyridine)platinum(II), or alternatively [SP-4-3]-ammine(dichloro)(2-methylpyridine)platinum(II). The compound is a square planar complex of divalent platinum that is tetracoordinate and has three different ligand types. Two ligands are anionic, and two are neutral; therefore as the platinum in picoplatin carries a +2 charge, picoplatin is itself a neutral compound and no counterions need be present. The name “picoplatin”, referring to the presence of α-picoline (2-methylpyridine) in the molecule, is the United States Adopted Name (USAN), the British Approved Name (BAN), and the International Nonproprietary Name (INN) for this material. Picoplatin is also referred to in the literature as NX473, ZD0473, and AMD473, and is disclosed in U.S. Pat. Nos. 5,665,771, 6,518,428, and U.S. Ser. No. 10/276,503.

Picoplatin is been provided to patients in solution by intravenous (IV) administration. Picoplatin under standard conditions is a solid, and has only sparing solubility in water. The relatively low solubility of picoplatin in water (about 1 mg/mL) necessitates that substantial volumes of liquid be delivered intravenously to provide a patient with total doses in the range of 100 mg and more (i.e., at a concentration of 0.5 mg/mL, some 200 mL of liquid must be introduced by IV infusion to provide a 100 mg dose). As typical human dosages for cancer patients can be on the order of several hundred milligrams per administration, and may be repeated every few weeks, substantial volumes of liquid must be delivered to the patient for each administration of the substance by the IV route. Intravenous administration is thus undesirable due to the need for needle insertion into a vein, and the relatively prolonged periods over which the patient must be immobile to allow for infusion of the relatively large volumes of the picoplatin solutions. Picoplatin is orally bioavailable, but its low solubility in water poses an obstacle to the preparation of effective oral dosage forms.

Picoplatin has also been found to be hydrolytically unstable, particularly under certain storage conditions, undergoing conversion to two isomeric species designated Aquo 1 and Aquo 2, the structures of which are shown below:

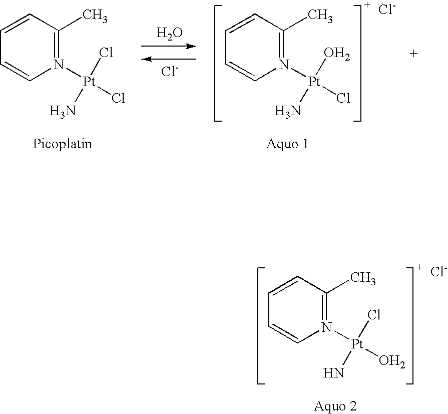

### SUMMARY

The present invention provides formulations for picoplatin adapted for oral administration to a cancer patient. The formulations comprise (a) a self-emulsifying formulation containing picoplatin, (b) a plurality of stabilized picoplatin nanoparticles, (c) a picoplatin solid dispersion in a water-dispersible matrix material, (d) a nanoparticulate picoplatin suspension in a medium chain triglyceride or a fatty ester, or any combination thereof. The formulation can provide improved oral availability of the picoplatin relative to an equivalent dose of solid picoplatin such as in a tablet, or to an equivalent dose of picoplatin in a simple solution such as in water or normal saline solution, that is orally ingested.

An embodiment of the invention concerns a self-emulsifying formulation of picoplatin. The self-emulsifying formulation includes picoplatin, an oil and an emulsifier, and, optionally, a first solvent. Examples of the oil include a medium chain triglyceride, a fatty ester, or an edible vegetable oil, such as peanut oil, cottonseed oil, or soybean oil. The emulsifier can be a lecithin, a polyethylene glycol (PEG), or a surfactant, or any combination thereof.

In another embodiment according to the invention, a method of preparing a self-emulsifying formulation of picoplatin using a solvent method is provided. The method includes dissolving picoplatin in a first solvent other than DMSO to provide a picoplatin solution, then adding an oil, and an emulsifier comprising a lecithin, a PEG, or a surfactant, or any combination thereof; then, adding a second solvent to dissolve the picoplatin solution, the oil, and the emulsifier, providing a substantially homogeneous second solution; then, evaporating at least the second solvent and, optionally, the first solvent, from the homogeneous solution to provide the self-emulsifying formulation.

Another embodiment of the invention concerns a formulation that includes a plurality of stabilized picoplatin nanoparticles. The picoplatin nanoparticles, having an average particle diameter of less than about one micron, are stabilized to inhibit aggregation, and can be stabilized with casein, a caseinate, or lecithin, or any combination thereof.

In another embodiment, a method of preparation of a formulation of stabilized picoplatin nanoparticles is provided, the method comprising mixing a stabilizer and an aqueous medium under high-shear conditions or microfluidization conditions to obtain a uniform dispersion, then adding solid picoplatin, and then mixing until an average particle size of the solid picoplatin is less than about one micron or until crystalline particles are substantially absent, or both, to provide a suspension of the stabilized picoplatin nanoparticles. The suspension can further be dried, such as by freeze-drying, to obtain a substantially dry picoplatin formulation.

Another embodiment of the invention concerns a picoplatin solid dispersion in a water-dispersible matrix material. The water-dispersible matrix material can comprise a PEG-ylated mono- or diglyceride.

In another embodiment, a method of preparing a picoplatin solid dispersion in a water-dispersible matrix material using a melt method is provided, wherein the picoplatin is dissolved in a melt of the matrix material, which is then cooled to provide the solid dispersion.

In another embodiment, a nanodispersion of picoplatin in medium chain triglyceride (MCT) oil or in a fatty ester, for example ethyl oleate, is provided. In an embodiment, a method of preparing the picoplatin nanodispersion in an MCT oil or in a fatty ester is provided.

In another embodiment, an oral picoplatin formulation comprising a substantially water-soluble capsule shell, the shell enclosing a formulation comprising a substantially dry, finely particulate material comprising, in admixture, about 10 to 60 wt % picoplatin, wherein the picoplatin is, in physical form, particulates of less than about 10 microns average particle diameter, in admixture with a substantially water-soluble, water-dispersible, or water-absorbing carbohydrate and an effective amount of up to about 5 wt % of a lubricant (or “glidant”), is provided.

In another embodiment, an oral picoplatin formulation, wherein the dosage form comprises a solid core comprising about 10 to 60 wt % particulate picoplatin wherein the picoplatin is a particulate of less than about 10 microns average particle diameter, about 40-80 wt % of a filler comprising a substantially water-soluble, water-dispersible, or water-absorbing carbohydrate, and an effective amount of up to about 5 wt % of a lubricant, and optionally a dispersant; and a continuous coating on the outer surface of the core; wherein the core and/or the coating are substantially free of redox-active metal salts, is provided.

In various embodiments, the present invention provides a method of treating cancer comprising administering an oral formulation of the invention or an oral formulation prepared by a method of the invention to a patient afflicted by cancer, in an amount, at a frequency, and for a duration of treatment effective to provide a beneficial effect to the patient. The patient can be chemotherapy-naïve or the patient can have previously received chemotherapy and/or radiation therapy.

In various embodiments, the cancer can be lung cancer including small cell lung cancer (SCLC) and non-small cell lung cancer (NSCLC), kidney cancer, bladder cancer, renal cancer, stomach and other gastrointestinal (GI) cancers, mesothelioma, melanoma, peritoneal lymphoepithelioma, endometrial cancer, glioblastoma, pancreatic cancer, cervical cancer, testicular cancer, ovarian cancer, colorectal cancer, esophageal cancer, uterine cancer, endometrial cancer, prostate cancer, thymic cancer, breast cancer, head and neck cancer, liver cancer, sarcomas, including Kaposi's sarcoma, carcinoid tumors, other solid tumors, lymphomas (including non-Hodgkins lymphoma, NHL), leukemias, bone-associated cancers and other cancers disclosed in the patents and patent applications cited herein.

In various embodiments, an embodiment of the oral formulation can be administered repeatedly to a patient suffering from cancer, at a dose, in a frequency, and for a duration sufficient to provide a beneficial effect to the patent. The oral picoplatin formulation can be administered in conjunction with a second anticancer agent or anticancer therapy. For example, the oral formulation can be administered in conjunction with radiotherapy such as X-ray or ry-ray irradiation, particle beam irradiation, brachytherapy, or radioisotope therapy, for treatment of the cancer.

In various embodiments, the oral formulation can be administered with a second anticancer agent comprising a molecular entity such as a small molecule or a protein. The second anticancer agent can be included in the oral formulation and thus administered in a combination with the picoplatin, or the second anticancer agent can be administered separately from the picoplatin. If administered separately, it can be administered substantially concurrently, prior to, or after administration of the oral formulation. The second anticancer agent can be administered orally or parenterally, for example intravenously. Examples are provided hereinbelow, and can be termed non-platinum containing anti-cancer agents or platinum-containing anti-cancer agents. The second anticancer agent can be provided at doses, frequencies of administration, and over a duration of time in combination with picoplatin doses, frequencies of administration, and over a duration of time effective to provide a beneficial effect to the patient.

In another embodiment of the invention, the present formulation is provided as a kit; i.e., enclosed in packaging with instruction materials, such as paper labeling, a tag, a compact disk, a DVD, a cassette tape and the like, regarding administration of the formulation to a patient. For example, the instruction materials can comprise labeling describing/directing a use of the formulation that has been approved by a government agency responsible for the regulation of drugs.

### BRIEF DESCRIPTION OF THE FIGURES

FIG. 1 shows an HPLC calibration curve for picoplatin.

FIG. 2 shows an HPLC trace of 0.5 mg/mL picoplatin standard solution in normal saline.

FIG. 3 shows an HPLC trace of 0.5 mg/mL picoplatin solution stored in deionized water at 40 deg C. for 2 days.

FIG. 4 shows HPLC traces of, from the bottom up, 0.5 mg/mL picoplatin solution in pH 2, 3, 4, 5, 6 buffers, normal saline and deionized water, each stored for 2 days at 40° C.

FIG. 5 is a graph showing the solubility of picoplatin in neutral water and in buffers of various pH values.

FIG. 6 shows picoplatin recovery (% over initial) at 25° C. after 0, 1 and 2 days.

FIG. 7 shows picoplatin recovery (% over initial) at 40° C. after 0, 1 and 2 days.

FIG. 8 shows the stability over time of picoplatin in dimethylsulfoxide (DMSO) with added buffers at various pH values.

FIG. 9 shows representative chromatograms of picoplatin in N-methyl-pyrrolidone (NMP) at 25° C. for 4 hours. From top down: 0.5 mg/mL in 100% NMP, 0.5 mg/mL in 80% NMP in normal saline, 0.5 mg/mL in 50% NMP in normal saline, 0.5 mg/mL in 20% NMP in normal saline, and 0.5 mg/mL standard in normal saline.

FIG. 10 shows HPLC chromatograms of Picoplatin in reconstituted solutions. The reconstituted solutions were obtained by adding normal saline to lyophilized picoplatin from various NMP solvents. From top down: from 100% NMP, from 80% NMP in normal saline, from 50% NMP in normal saline, from 20% NMP in normal saline, and from normal saline.

FIG. 11 shows a thermogravimetric/differential thermal analysis (TG/DTA) scan of micronized picoplatin powder.

FIG. 12 shows a thermogravimetric/differential thermal analysis (TG/DTA) scan of TG/DTA of F50 Picoplatin nanoparticles in sodium caseinate.

FIG. 13 shows representative HPLC chromatograms of picoplatin nanoparticles. From the top down: 0.5 mg/mL nanoparticles in normal saline and 0.5 mg/mL picoplatin standard in normal saline. One unknown peak at 5.5 min (not Aquo #1).

FIG. 14 shows representative HPLC Chromatograms after hot melt in Gelucire 50/15. From top down: 0.5 mg/mL picoplatin standard in normal saline and 0.5 mg/mL F51 in normal saline.

FIG. 15 shows a representative DSC for Picoplatin in Gelucire 50/15 hot melt. From top down: Gelucire 50/15, 5% picoplatin in Gelucire 50/15 hot melt, and picoplatin API.

FIG. 16 shows a representative DSC for Picoplatin in hot melt. From top down: 5% picoplatin in Gelucire 50/15, 6% picoplatin in Gelucire 50/15 and 5% in Compritol 888 ATO.

FIG. 17 shows HPLC traces, from the top down: 0.5 mg/mL standard in neutral saline, F73-picoplatin in MCT, F74-picoplatin in MCT and PL90G, and F75-picoplatin in MCT and Polysorbate 80.

FIG. 18 shows zoomed-in views of the HPLC traces of FIG. 17. From the top down: 0.5 mg/mL standard in normal saline, F73-picoplatin in MCT, F74-picoplatin in MCT and PL90G, and F75-picoplatin in MCT and Polysorbate 80.

FIG. 19 shows representative HPLC chromatograms From top down: 0.5 mg/mL standard in normal saline, F77-picoplatin in Ethyl Oleate and PL90, F80-picoplatin in MCT, PL90G and normal saline.

FIG. 20 shows representative HPLC chromatograms, enlarged. From top down: 0.5 mg/mL standard in neutral saline, F77-picoplatin in Ethyl Oleate and PL90, F80-picoplatin in MCT, PL90G and normal saline.

FIG. 21 shows representative HPLC Chromatograms. From top down: 0.5 mg/mL picoplatin standard in normal saline and 0.5 mg/mL F81-picoplatin in PL90 and EO in normal saline.

FIG. 22 shows representative HPLC chromatograms, enlarged. From top down: 0.5 mg/mL picoplatin standard in normal saline and 0.5 mg/mL F81-picoplatin in PL90 and EO in normal saline.

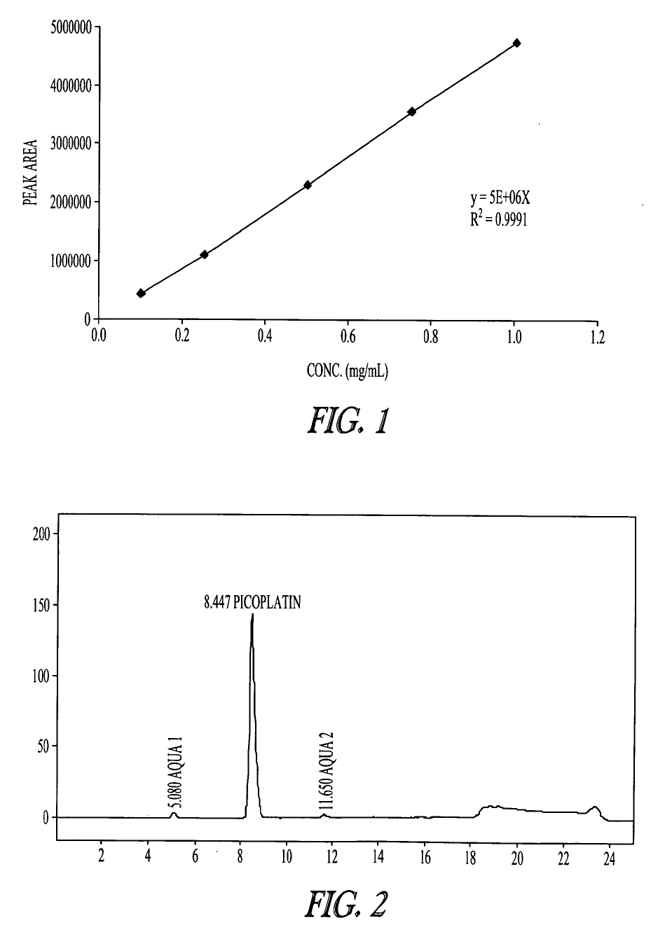

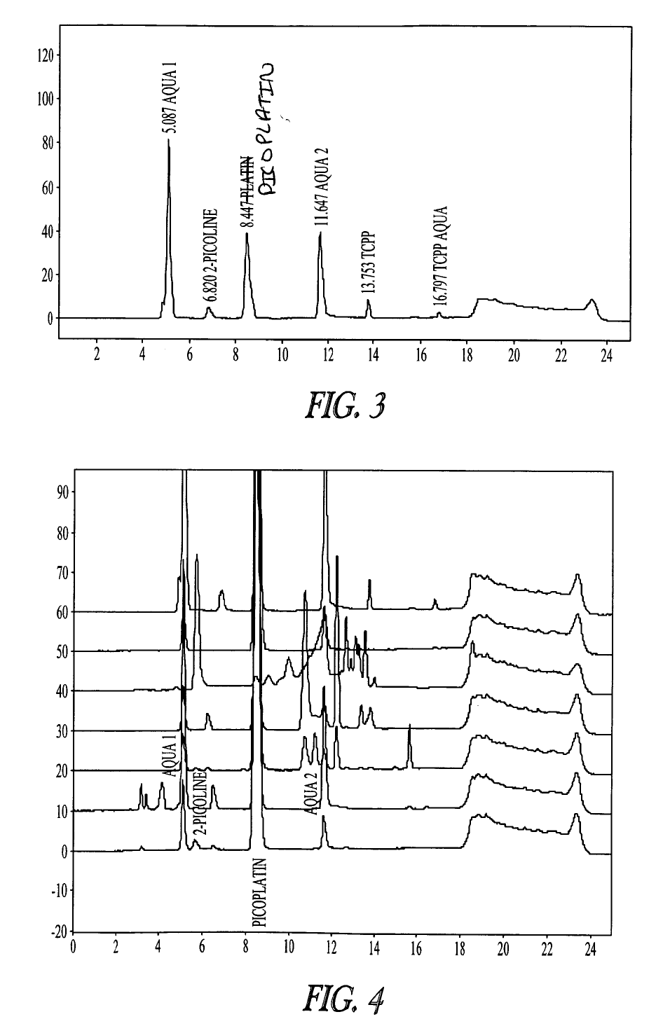

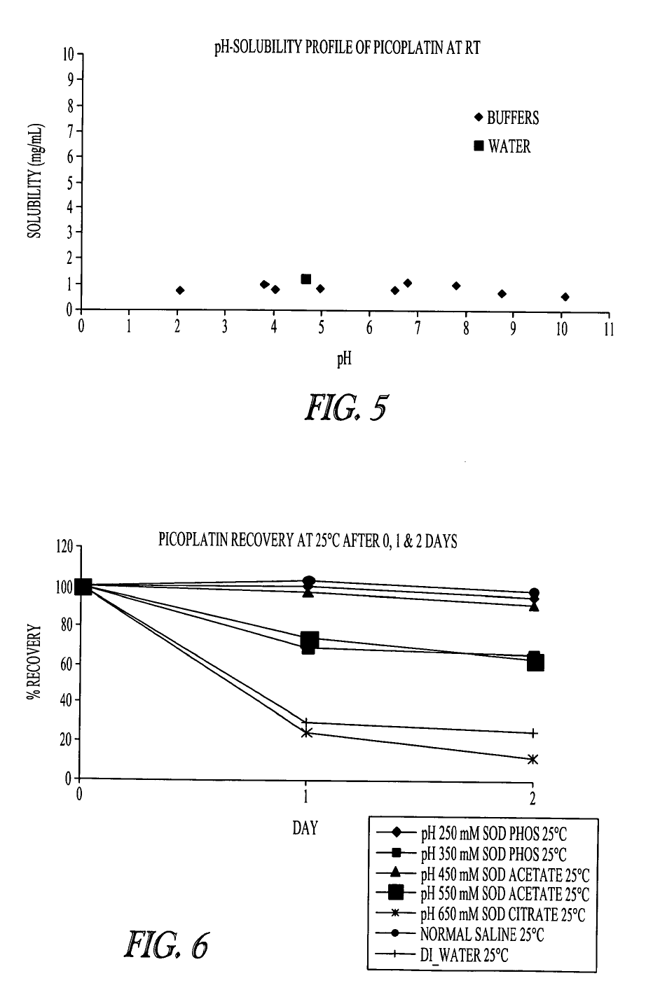

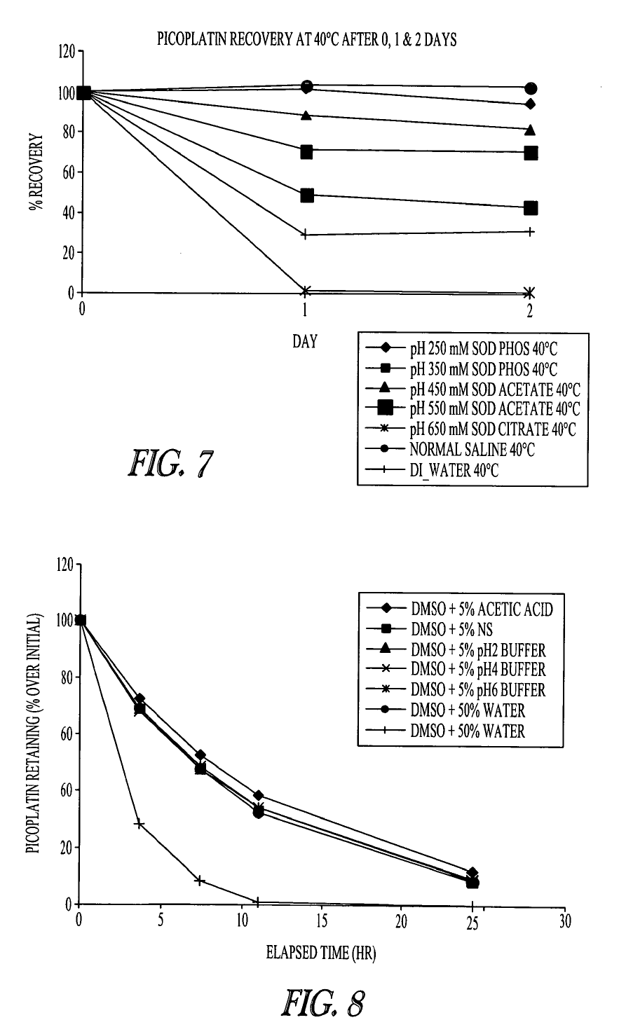

### DEFINITIONS

As the term is used herein, “picoplatin” refers to the organoplatinum anticancer drug, the structure of which is provided above, including any solvate, hydrate, or crystalline polymorph thereof, in solid form, or in solution or dispersion.

A “formulation” as the term is used herein is a composition of matter including picoplatin and other components, such as excipients, stabilizers, dispersants, surfactants, and the like.

“Self-emulsifying” refers to a property of a formulation wherein upon contacting the formulation with an aqueous medium, such as in the gastro-intestinal tract of a patient, the formulation spontaneously forms an emulsion.

“Nanoparticles” are solid particles of an average particle diameter of less than about 1 micron (micrometer, μm). One micron is 1,000 nanometers (nm).

“Stabilized” nanoparticles are picoplatin nanoparticles coated with a stabilizing material and having a reduced tendency for aggregation and loss of dispersion with respect to nanoparticles of picoplatin without a stabilizing coating.

“Casein” is a milk-derived protein that typically is globular in aqueous dispersion, as is well known in the art. A “caseinate” is a salt form of casein wherein carboxylate groups in the protein are present in ionized form, such as the sodium salts (“sodium caseinate”).

“Microfluidization” is a technique for preparing dispersions of fine particles in a liquid medium wherein coarser particles are comminuted in the presence of the liquid medium.

“High-shear mixing” is a technique for preparing dispersions of fine particles in a liquid medium wherein high-shear conditions comminute coarser particles into finer ones in the presence of the liquid medium.

A “solid dispersion” as the term is used herein refers to a dispersion of solid picoplatin in a solid or semi-solid matrix. The solid dispersion can be formed in a liquid or melt phase wherein the final mixture solidifies into the solid or semi-solid form.

“Water-dispersible” means that a solid or semi-solid material can be suspended in an aqueous medium and does not spontaneously phase separate from the aqueous medium. “Water-dispersible” includes “water-soluble”, referring to a solid or semi-solid material that completely dissolves in the aqueous medium to form a homogeneous solution. A “matrix” as the term is used herein refers to an organic material, that is at least dispersible in water, that is solid at about room temperature or about human body temperature, in which picoplatin can be dispersed.

An “oil” as the term is used herein refers to an organic liquid, which is water-insoluble, or at least only partially water-soluble, that can form a separate phase in the presence of water. An example of an “oil” is a glyceride such as a medium chain triglyceride, or a medium chain mono- or di-glyceride, or castor oil. Another example of an oil is a fatty ester. A fatty ester refers to an alkyl ester of a fatty acid. An example is ethyl oleate. “MCT oil” refers to medium chain triglyceride oil. Examples include the MCT oil sold under the Miglyol trademark, such as Miglyol 912, a caprylate/caprate (octanoate/decanoate triglyceride).

A “nanodispersion” is a dispersion of picoplatin particles of less than 1 μm average particle diameter in a liquid, for example in MCT oil or in a fatty ester.

A “lecithin” as the term is used herein is a mixture of triglycerides, glycolipids, and phospholipids such as phosphatidylcholine, as is well-known in the art. Lecithins can be derived from eggs or from soy beans. A high-phosphatidylcholine lecithin is a lecithin with a relatively high phosphatidyl-choline (PC) content. A low-phosphatidylcholine lecithin is accordingly a lecithin with a relatively low PC content.

A “surfactant” as the term is used herein is a substance that reduces interfacial surface tension between immiscible liquids such as oil and water, reduces surface tension of a water drop, and exhibits other surface-active properties as are well known in the art.

The term “weight average molecular weight” is well known in the art and characterizes an average molecular weight of a polydisperse sample of a polymer.

A “PEG” or a “polyethyleneglycol” is a polymeric material composed of repeating—CH₂CH₂O— units, wherein there are two or more units. Thus, diethyleneglycol and all higher polymers are polyethyleneglycols within the meaning herein. A polyethyleneglycol can have a free OH group at either terminus or at both termini, or can alternatively include other groups such as an ether group at one or both ends, for example a methyl ether CH₃O—(CH₂CH₂O)ₙ—OCH₃. Such an ether-terminated PEG can also be referred to as a “polyethyleneglycol ether”. PEG-400 is a PEG with a weight average molecular weight of about 400 DA. PEG-8000 is a PEG with a weight average molecular weight of about 8000 DA. A compound can be “PEG-ylated”, meaning that it bears at least one PEG group, which can be introduced in a variety of ways, such as by polymerization of ethylene glycol initiated by the compound, or coupling of the compound with a preformed PEG. For example, Gelucire® is a PEG-ylated fatty acid monoglyceride, meaning that a glycerol moiety bears a single fatty acid moiety and PEG moieties on one or both of the remaining free hydroxyl groups.

A “dipolar aprotic solvent” is a solvent not containing a source of protons in aqueous solution (an example of a protic solvent is ethanol) that also is polar in character and is typically at least partially soluble in water. Examples of aprotic solvents are DMF, NMP, DMSO, DMAC, and the like. “DMSO” is dimethylsulfoxide. “NMP” is N-methylpyrrolidone. “DMF” is N,N-dimethyl-formamide. “DMAC” is N,N-dimethylacetamide.

“Labrasol®” is a mixture composed of about 30% mono-, di-, and triglycerides of C8 and C10 fatty acids, 50% of mono- and di-esters of polyethyleneglycol (PEG 400), and 20% of free PEG 400. Labrasol® has surfactant properties.

“Cremophor RH 40®” is a nonionic solubilizer and emulsifying agent obtained by reacting 45 moles of ethylene oxide with 1 mole of hydrogenated castor oil. The main constituent of Cremphor RH 40® is glycerol polyethylene glycol oxystearate, which, together with fatty acid glycerol polyglycol esters, forms the hydrophobic part of the product. The hydrophilic part consists of polyethylene glycols and glycerol ethoxylate.

“Cremophor ELP®” is a nonionic solubilizer made by reacting castor oil with ethylene oxide in a molar ratio of 1:35.

“Gelucire®” including Gelucire 44/14 (CAS RN 121548-04-7) and Gelucire 50/13 (CAS RN 121548-05-8) are fatty acid glycerides bearing polyethyleneglycol (PEG) groups. For example, Gelucire 44/14 is a PEG-ylated glyceride of lauric acid; Gelucire 50/13 is a PEG-ylated glyceride of stearic acid. The numbers after the word Gelucire refer to the melting point in ° C. and the hydrophilic-lipophilic balance (HLB) value respectively. Gelucire compounds are PEG-ylated with PEG 1500 (polyethyleneglycol of weight average molecular weight 1500 DA).

“Polysorbate 80” refers to sorbitan mono-9-octadecanoate poly(oxy-1,2-ethanediyl) derivatives; they are well known as complex mixtures of polyoxyethylene ethers used as emulsifiers or dispersing agents in pharmaceuticals.

“Phospholipon 90G” or “PL90G” (American Lecithin Products, Oxford, Conn.) is a tradename for lecithin, minimum 94% phosphatidylcholine for the manufacture of liposomes. “Phospholipon 90H” or “PL90H” is a hydrogenated PL90G. The term “PL90” refers to either one of these materials.

“Vitamin E TPGS” refers to the compound D-alpha-tocopheryl polyethylene glycol 1000 succinate.

“Compritol 888” refers to glyceryl behenate. A “behenate” is an ester of docosanoic acid, as is well known in the art.

“Poloxamer 188” (CAS RN 9003-11-6) is a Polyethylene-Polypropylene Glycol copolymer of the formula HO(C₂H₄O)ₐ(C₃H₆O)_{b}(C₂H₄O)ₐH with a weight average molecular weight of about 8400

“SPAN 60” refers to sorbitan monostearate.

“Kollidon K90” (Hoechst, Germany) refers to a polyvinylpyrrolidone with a molecular weight of about 90,000.

“Miglyol 812” (Sasol Germany GmbH, Witten, Germany) refers to a medium chain triglyceride wherein the acid moieties are caprylic and capric acid. Miglyol is a trademark identifying the source of this and other varieties of MCT oil.

“Administering” or “administration” refers to providing a medicinal compound to a patient in need thereof. A “dose” is the amount of the active pharmaceutical ingredient (API), in this case picoplatin, that is provided in a single administration. A “frequency” of administration refers to how often the medication is given when repeated doses are prescribed; for example, the medication can be administered daily. A “duration” refers to the period of time over which repeated doses are administered; for example, the picoplatin can be administered for a duration of two weeks.

A “second medicament comprising an anticancer medicament” can include, without limitation, a taxane (e.g.: paclitaxel (Taxol®) or docetaxel (Taxotere®), a tyrosine kinase and/or a growth factor receptor inhibitor such as a VEGFR inhibitor (e.g.: monoclonal antibodies such as: bevacizumab (Avastin®), trastuzumab (Herceptin®), panitumumab (Vectibix®) or cetuximab (Erbitux®)); a cephalotaxine analog (e.g.: topotecan (Hycamtie); irinotecan; 9-aminocamptothecin; Rubitecan®; Exatecan®; XR-5000, XR-11576); an anti-metabolite (e.g.: capecitabine (Xeloda), gemcitabine, 5-FU with or without leucovorin, S1 (gimeracil/oteracil/tegafur), tegafur/uracil, methotrexate, or a thymidylate synthease inhibitor (Tomudex®, ZA9331, LY231514 (pemetrexed))); a protein kinase inhibitor (e.g.: sorafenib (Nexavar®), dasatinib (Sprycel®), gefitnib (ZD1839, Iressa®), imatinib (Gleevac®), lapatinib (Tykerb®), cediranib, also known as AZD2171 (Recentin®), erlotinib (Tarceva®) or sunitinib (Sutent®)); an anthracyclin (e.g.: amrubicin, doxorubicin, liposomal doxorubicin, epirubicin, idarubicin, Doxil®); a Vinca alkaloid (e.g.: vinorelbine (Navelbine), vincristine, vinblastine, vindesine); a podophyllotoxin analog (e.g.: etoposide, teniposide); a growth factor inhibitor (e.g.: inhibitor of PDGF, endothelial GF, VEGF, EGF, or hepatocyte GF; for example an GF-binding antibody or a GF receptor-binding antibody); an inhibitor of cell cycle kinases (such as CDK-2, CDK-4, or CDK-6); a cytostatic agent (Tamoxifen, Toremifene, Raloxifene, Droloxifene, Iodoxyfene; megestrol acetate; an aromatase inhibitor such as Anastrozole (ZD1033), Letrazole, Vorazole, Exemestane; an antiandrogen such as Flutamide, Nulutamide, Bicalutamide, Cyproterone acetate; an LHRH agonist or antagonist such as Foserelin acetate or Luprolide; an inhibitor of testosterone dihyhdroreductase such as Finasetide, a metalloproteinase inhibitor such as Marimastat or a uPAR inhibitor); an alkylating agent (e.g.: melphalan, cyclophosphamide, ifosphamide, nitrosourea, carmustine, lomustine); or radiation therapy (e.g.: X-ray, γ-ray, particle beam, brachytherapy, radioisotope).

Alternatively, the additional medicament is a non-platinum containing agent, can be selected to treat a complication of the cancer, or to provide relief to a subject from at least one symptom of the cancer, for example, sirolimus or rapamycin (Rapamune®), dexamethasone (Decadron®), palonosetron HCl (Aloxi®), aprepitant (Emend), ondansetron (Zofran®), or granisetron (Kytril®).

Examples of anti-cancer medicaments that can be orally administered are listed in Table 1, below.

TABLE 1
Orally Administrable Agents

altretamine
anagrelide
anastrozole
(ZD1033)
bexarotene
bicalutamide
capecitabine
clodronic acid
cytarabine
ocfosfate
dasatinib
dutasteride
erlotinib
exemestane
fadrozole
finasteride
fludarabine
gefitinib
GMDP
HMPL 002
hydroxycarbamide
ibandronic acid
idarubicin
imatinib
lapatinib
lenalidomide
letrozole
osaterone
polysaccharide K
prednimustine
S1
(gimeracil/oteracil/tegafur)
sobuzoxane
sorafenib
sunitinib
tamibarotene
tamoxifen
tegafur/uracil
temozolomide
thalidomide
topotecan
toremifene
treosulfan
trilostane
ubenimex
vinorelbine
vorinostat

Orally active anticancer agents that can be administered include altretamine (Hexylen®), an alkylating agent; capecitabine (Xeloda®), an anti-metabolite; dasatinib (Sprycel®), a TK inhibitor; erlotinib (Tarceva®), an EGF receptor antagonist; gefitinib (Iressa®), an EGF inhibitor; imatinib (Gleevec), a TK inhibitor; lapatinib (Tykerb®), an EGFR inhibitor; lenalidomide, (Revlimid®), a TNF antagonist; sunitinib (Sutent®), a TK inhibitor; S-1 (gimeracil/oteracil/tegafur), an anti-metabolite; sorafenib (Nexavar®), an angiogenesis inhibitor; tegafur/uracil (UFT®, Uftoral®), an anti-metabolite; temozolomide (Temodar®), an alkylating agent; thalidomide (Thalomid®), an angiogenesis inhibitor; topotecan (Hycamtin® for injection or Oral Hycamtin®), vinorelbine (Navelbine), an anti-mitotic; cediranib (AZD2171, Recentie), a VEGF inhibitor; and/or vorinostat (Zolinza®), a histone deacetylase inhibitor.

As the term is used herein, “radiation” or “radiotherapy” refers to the treatment of cancer patients with various forms of ionizing radiation, which acts to a great extent on dividing cells by interfering with DNA replication and cell division. The three main types of radiotherapy are external beam radiotherapy (EBRT or XBRT) or teletherapy, brachytherapy or sealed source radiotherapy and unsealed source radiotherapy. The differences relate to the position of the radiation source; external is outside the body, while sealed and unsealed source radiotherapy has radioactive material delivered internally. External beam radiotherapy can involve beams of photons, such as X-rays, or beams of particles, such as protons. External beam radiotherapy can involve either total body irradiation or the use of multiple focussed beams to concentrate the energy in a defined volume of body tissue. Brachytherapy involves implantation of sealed sources of various radioisotopes within body tissues, such that the sources can be removed after a period of time. The type of radiation emitted depends on the identity of the radioisotope included in the sealed source, and can be photon (X-ray) or particle (e.g., beta particle). When unsealed sources are used, e.g., radiolabeled antibodies or the like, the nature of the radiation again depends on the identity of the radioisotope used, but due to the fact that there is no containment, particles of shorter range such as alpha particle and Auger electrons can be used effectively. However, since unsealed sources typically cannot be removed surgically, the radioisotopic form must be one that can be excreted, or else decays, within an appropriate time frame. Examples of useful isotopes include ⁹⁰Y, ¹³¹I, and ¹⁷⁷Lu.

### DETAILED DESCRIPTION OF THE INVENTION

The present invention concerns formulations of the anticancer drug picoplatin adapted for oral administration to a cancer patient, and to methods of preparation of the formulations. In an embodiment of the invention, a self-emulsifying formulation provides the picoplatin dissolved in a one-phase oleaginous vehicle, which forms an emulsion upon exposure to an aqueous medium in the gastrointestinal tract, and delivers picoplatin in emulsified oil droplets with a potential for better intestinal absorption into the bloodstream. A self-emulsifying formulation can include an oil (oleaginous vehicle) along with dispersants and surfactants that assist in the self-emulsification properties of the formulation. Once orally ingested by a patient, the formulation can emulsify in the gastrointestinal tract. The formulation can provide improved oral availability of the picoplatin relative to an equivalent dose of solid picoplatin such as in a tablet, or to an equivalent dose of picoplatin in a simple solution such as in water or normal saline solution, that is orally ingested.

An embodiment of the self-emulsifying picoplatin formulation can include an oil, and an emulsifier including a lecithin, a surfactant, a PEG, or any combination thereof. Preferably, the self-emulsifying formulation includes at least about 10% w/w of the picoplatin, although it can include lesser amounts of picoplatin, for example, 5% w/w of the picoplatin. The inventive self-emulsifying formulation can also include a first solvent in which picoplatin is at least sparingly soluble, provided that the first solvent is not DMSO. As disclosed hereinbelow, picoplatin is unstable in DMSO, perhaps due to oxidation of the picoplatin by the DMSO. The first solvent can be a dipolar aprotic solvent, a polyethylene glycol, or a polyethyleneglycol ether, a polyethyleneglycol derivative of a mono- or a di-glyceride, or any combination thereof. The dipolar aprotic solvent can be NMP. Preferably the dipolar aprotic solvent, particularly if it is NMP, is substantially free of amine contaminants.

For example, the first solvent can be a polyethyleneglycol derivative of a mono- or a di-glyceride, such as Gelucire 40/14® or Gelucire 50/13®. The picoplatin can be dissolved in the Gelucire held above Gelucire's melting point, i.e., 40° C. for Gelucire 40/14, or 50° C. for Gelucire 50/13. The solution of the picoplatin in the melted Gelucire can then be mixed with other components in the second solvent to form a substantially homogenous second solution. The Gelucire (polyethyleneglycol derivative of a mono-glyceride, i.e., a PEG-ylated monoglyceride) is itself a surfactant; thus mixing the Gelucire solution of the picoplatin with the oil in the second solvent, followed by removal of the second solvent, can provide the self-emulsifying formulation of the invention, wherein the Gelucire serves both as the first solvent and as the emulsifier. Alternatively, lecithin, PEG, another surfactant, or any combination thereof, can also be mixed with the second solvent to provide a substantially homogeneous solution, from which the second solvent is removed to provide the present self-emulsifying formulation.

The self-emulsifying formulation includes an oil, wherein the oil is a medium chain triglyceride, castor oil, a medium chain mono-glyceride, a medium chain di-glyceride, an edible vegetable oil such as peanut oil, cottonseed oil, or soybean oil, or any combination thereof. Alternatively, the oil can be other than a glyceride; for example, the oil can be a hydrocarbon oil or a silicone oil.

The self-emulsifying formulation includes an emulsifier. For example, the emulsifier can contain a lecithin. The lecithin can be a high phosphatidyl-choline content lecithin, a low phosphatidylcholine content lecithin, or any combination thereof.

The emulsifier can also include a surfactant, such as Labrasol® (a mixture of glycerides and PEG-ylated materials), Cremophor RH408 (a PEG-ylated glyceride), Cremophor ELP® (a PEG-ylated glyceride), Gelucire 44/14® (a PEG-ylated glyceride), Polysorbate 80 HP® (a PEG-ylated fatty ester of sorbitan), or Vitamin E TPGS (a PEG-ylated tocopherol succinate), or any combination thereof. Gelucire can be both the first solvent and the emulsifier of the inventive self-emulsifying formulation.

The present self-emulsifying formulation can contain a PEG, such as PEG-400. PEG compounds are typically water-soluble, but also can stabilize hydrophobic materials in aqueous media.

A method of preparation of the self-emulsifying formulation is likewise provided as an embodiment of the invention herein. For example, the formulation can be prepared by dissolving picoplatin in a first solvent other than DMSO to provide a picoplatin solution, then adding an oil, and an emulsifier comprising a lecithin, a PEG, or a surfactant, or any combination thereof; then, adding a second solvent to dissolve the picoplatin solution, the oil, and the emulsifier, providing a substantially homogeneous second solution; then, evaporating at least the second solvent and, optionally, the first solvent, from the homogeneous solution to provide the self-emulsifying formulation.

The first solvent can be a dipolar aprotic solvent, a polyethylene glycol, or a polyethyleneglycol ether, a polyethyleneglycol derivative of a mono- or di-glyceride, or any combination thereof. The dipolar aprotic solvent can be NMP. Preferably the dipolar aprotic solvent, particularly if NMP, is substantially free of amine contaminants. DMSO is not suitable as the first solvent, due to the instability of picoplatin in DMSO. A solution of a preselected amount of picoplatin for the batch formulation being prepared is dissolved in the first solvent, then the emulsifier is added. The emulsifier can include a lecithin, a PEG, a surfactant, or any combination thereof. The oil can be a medium chain triglyceride, castor oil, a medium chain mono-glyceride, a medium chain di-glyceride, or any combination thereof. The lecithin can be a high phosphatidylcholine content lecithin, a low phosphatidylcholine content lecithin, or any combination thereof. The PEG can be PEG-400. The surfactant can be Labrasol, Cremophor RH40, Cremophor ELP, Gelucire 44/14, Polysorbate 80 HP, or Vitamin E TPGS, or any combination thereof.

Then, a second solvent is added to provide a substantially homogenous second solution, at or near room temperature, although some heating can be used to assist dissolution of all components. Then, the second solvent is removed from the homogenous solution. A suitable second solvent is ethanol, which can be removed under reduced pressure at or near room temperature, although elevated temperatures can also be used. The evaporation can continue such that the first solvent is also removed, although the first solvent or portions of it can remain in the formulation. The residue is a self-emulsifying formulation of the invention, which can be liquid, solid or semi-solid. This material can be filled into hard or soft gelatin capsules for administration to a patient. The self-emulsifying formulation is adapted to aid in dissolution of the picoplatin in the gastrointestinal (GI) tract of the patient, and thus provide for enhanced uptake into the bloodstream compared to the same dose of picoplatin administered as a pure solid.

In another embodiment of the invention, a stabilized nanoparticle preparation of picoplatin is provided that possesses a greatly increased surface area and thus an improved dissolution rate relative to solid crystalline picoplatin. The picoplatin nanoparticles are stabilized with organic materials. For example, the picoplatin nanoparticles can be stabilized with casein, a caseinate, or lecithin, or any combination thereof. Casein and caseinates are proteins found in milk that serve to stabilize butterfat droplets in the aqueous medium. In the present stabilized nanoparticle formulation, the casein or caseinates, or both, can stabilize the sub-micron size picoplatin particles and inhibit re-aggregation of the particles. Also, lipid compositions such as lecithin can be used to stabilize the picoplatin nanoparticles. Preferably, the formulation contains at least about 10% w/w of the picoplatin on a dry weight basis, although the formulation can include a lesser amount of picoplatin, for example, at least about 5% w/w of picoplatin, on a dry weight basis, or an intermediate weight. The formulation can provide improved oral availability of the picoplatin relative to an equivalent dose of solid picoplatin such as in a tablet, or to an equivalent dose of picoplatin in a simple solution such as in water or normal saline solution, that is orally ingested.

The picoplatin nanoparticles can be prepared by a process comprising high-shear mixing or microfluidization. Solid picoplatin, for example picoplatin in crystalline form, can be mixed in an aqueous medium with a stabilizer such as casein, using microfluidization conditions or high-shear conditions, until the average particle diameter of the solid picoplatin is less than about one micron as determined by laser light scattering spectroscopy, or, alternatively, until crystalline picoplatin is observed to be largely absent using an optical microscope with a polarized light filter lens. The average particle diameter can be even smaller; for example the picoplatin nanoparticles can have an average particle diameter of less than about 0.5 micron; of less than about 0.25 micron; or of less than about 0.15 micron.

An embodiment of the invention also provides a method of preparation of the stabilized picoplatin nanoparticles. The method includes mixing a stabilizer and an aqueous medium under high-shear conditions or microfluidization conditions to obtain a uniform dispersion, then adding solid picoplatin, and then continuing mixing under these conditions until an average particle size of the picoplatin is less than about one micron or until crystalline particles are substantially absent, or both, to provide a suspension of the stabilized picoplatin nanoparticles. The stabilizer can be casein, a caseinate, or a lecithin. The average picoplatin particle diameter can be less than about 1 micron, or less than about 0.5 micron, or less than about 0.25 micron, or less than about 0.15 micron.

The suspension of stabilized picoplatin nanoparticles can then be dried to provide a solid material, for example by freeze-drying, to provide a substantially dry solid. By this method, a solid formulation that can be filled into gelatin capsules for oral administration to a patient can be obtained. The picoplatin content of the substantially dry solid can be at least about 10% w/w, or at least about 5% w/w.

In another embodiment of the invention, a dispersion of solid picoplatin in a solid water-dispersible material (matrix) is provided. The inventive solid dispersion can be prepared by a process comprising dispersing of the picoplatin in a melt of the water-dispersible matrix material that then is cooled and solidified. Preferably, the formulation contains at least about 10% w/w of the picoplatin, although the formulation can include a lesser amount of picoplatin, for example, at least about 5% w/w of picoplatin. The water-dispersible matrix material can include Gelucire 50/13, Gelucire 44/14, Poloxamer 188, SPAN 60, PEG-8000, Kollidon K-90, Vitamin E TPGS, or Compritol 888, or any combination thereof, definitions of which are provided herein. The Gelucire and Compritol materials are PEG-ylated glycerides of fatty acids. Poloxamer is a polyethyleneglycol-polypropyleneglycol copolymer. Span is a monostearate ester of sorbitan, and Kollidon is a poly-vinylpyrrolidone. Vitamin E TPGS is a PEG-ylated toxopherol succinate.

The water-dispersible matrix material is at least dispersible in water, not phase-separating spontaneously, and can be completely water-soluble. The matrix material is preferably a solid at about 20° C. to about 37° C. The melt of the water-dispersible matrix material can be held at a temperature of about 40° C. to about 160° C. during dispersion of the solid picoplatin. The step of dispersing the picoplatin in the melt can involve dissolving the picoplatin in the melt to provide a homogenous melt. The homogeneous melt can include Gelucire 50/13, Gelucire 44/14, Compritol 888, or Vitamin E TPGS. The melt is then cooled and solidified to provide the inventive solid dispersion. The formulation can provide improved oral availability of the picoplatin relative to an equivalent dose of solid picoplatin such as in a tablet, or to an equivalent dose of picoplatin in a simple solution such as in water or normal saline solution, that is orally ingested.

In an embodiment of the invention, a nanoparticulate picoplatin suspension in a medium chain triglyceride (MCT oil) or in a fatty ester is provided. The nanoparticulate picoplatin comprises picoplatin particles of less than 1 micron average particle diameter, suspended in the MCT oil or fatty ester. The nanoparticulate picoplatin can make up about 20% up to about 70% by weight of the composition. The MCT oil can be a triglyceride ester of a medium chain fatty acid, or of a combination of different medium chain fatty acids. For example, the MCT oil can be tricaprylglyceride (trioctanoylglyeride) or can be a mixed caprylic/capric (octanoyl/decanoyl) glyceride. All three glycerin hydroxyl groups are acylated in the MCT oil. An example of an MCT oil is a Miglyol brand (Sasol) MCT oil, such as Miglyol 812). Alternatively, the nanoparticulate picoplatin suspension can include a fatty ester. An example is ethyl oleate. The suspension can further contain a lecithin, i.e., a phospholipid. An example is the brand Phospholipon 90G (American Lecithin). The suspension can further contain a sugar ester surfactant, such as a sorbitan ester. An example is sorbitan mono-9-octadecanoate PEG ether (sold under the brand name Sorbate 80).

An embodiment of the invention provides a method of preparation of the nanoparticulate picoplatin suspension comprising contacting the picoplatin in bulk form and the MCT oil or fatty ester, then mixing under high shear conditions until the average picoplatin particle diameter is 1 micron or less. A lecithin, a Sorbate-type surfactant, or both can also be present during the high shear mixing, or can be added subsequently. In an embodiment, following the high shear mixing, the solid picoplatin nanoparticulate form can be allowed to settle, or can be settled by centrifugation, and a portion of the supernatant liquid removed to provide a nanoparticulate picoplatin suspension with a higher picoplatin content than prior to removal of some of the supernatant liquid.

In another embodiment, an oral picoplatin formulation comprising a substantially water-soluble capsule shell, the shell enclosing a formulation comprising a substantially dry, finely particulate material comprising, in admixture, about 10 to 60 wt % picoplatin, wherein the picoplatin is, in physical form, particulates of less than about 10 microns average particle diameter, in admixture with a substantially water-soluble, water-dispersible, or water-absorbing carbohydrate and an effective amount of up to about 5 wt % of a lubricant (or “glidant”), is provided. The capsule shell is preferably composed of a biodegradable and/or digestible material, such as hard or soft gelatin, PVA, polylactides, polyglycolic acids, and the like. The picoplatin preferably is a particulate having an average particle diameter of 1-5 microns. The picoplatin particulate can be micronized, for example by jet-milling, or can be a microcrystalline material, such as can be prepared by precipitation, or can be a particulate formed by a lyophilization process, or any combination of the three processes. The picoplatin particulate can be dispersed within substantially every particle of the powder of the formulation. The oral picoplatin formulation, can comprise a substantially dry powder comprising about 20 to 55 wt % picoplatin wherein the picoplatin is particulates of less than about 10 microns average particle diameter, a substantially water-soluble, water-dispersible, or water-absorbing carbohydrate, and an effective amount of up to about 5 wt % of a lubricant, enclosed within a substantially water-soluble capsule shell. The formulation can also comprise an effective amount of a dispersing agent.

In another embodiment, an oral picoplatin formulation, wherein the dosage form comprises a solid core comprising about 10 to 60 wt % particulate picoplatin wherein the picoplatin is a particulate of less than about 10 microns average particle diameter, about 40-80 wt % of a filler comprising a substantially water-soluble, water-dispersible, or water-absorbing carbohydrate, and an effective amount of up to about 5 wt % of a lubricant, and optionally a dispersant; and a continuous coating on the outer surface of the core; wherein the core and/or the coating are substantially free of redox-active metal salts, is provided. Preferably both the coating and the core are free of amounts of redox-active metals that can be deleterious to the picoplatin in vivo or in vitro (e.g., in storage). The coating forms a protective covering for the core, both protecting the contents from environmental degradation by oxygen, light, and reactive chemicals, and protecting persons handling the dosage form from the cytotoxic picoplatin. The coating can comprise gelatin, either hard or soft; a polymer, for example hydroxypropyl methyl cellulose; a sugar, for example sucrose; or any other non-toxic, water soluble material suitable for human consumption. The picoplatin particulate that has an average particle diameter of less than about 10 microns, preferably has an average particle diameter of less than about 7 microns, and more preferably has a particle size distribution such that about 90% of the individual particulates have a diameter of less than about 5 microns.

In various embodiments, the present invention provides a method for treating cancer comprising administering an inventive oral formulation or an oral formulation prepared by an inventive method to a patient afflicted by cancer, in an amount, at a frequency, and for a duration of treatment effective to provide a beneficial effect to the patient. The patient can be chemotherapy-naïve or the patient can have previously received chemotherapy.

The dose, dosage form, frequency, and duration of administration can be determined by the attending physician, based upon his or her knowledge and experience, the body weight, skin area, disease state, and physical condition of the patient, and any other factors that the physician may decide are relevant to selection of a dose, frequency of administration, and duration of time over which the formulation is administered to the patient.

In various embodiments, the cancer can be lung cancer including small cell lung cancer (SCLC) and non-small cell lung cancer (NSCLC), kidney cancer, bladder cancer, renal cancer, stomach and other gastrointestinal (GI) cancers, mesothelioma, melanoma, peritoneal lymphoepithelioma, endometrial cancer, glioblastoma, pancreatic cancer, cervical cancer, testicular cancer, ovarian cancer, colorectal cancer, esophageal cancer, uterine cancer, endometrial cancer, prostate cancer, thymic cancer, breast cancer, head and neck cancer, liver cancer, sarcomas, including Kaposi's sarcoma, carcinoid tumors, other solid tumors, lymphomas (including non-Hodgkins lymphoma, NHL), leukemias, bone-associated cancers and other cancers disclosed in the patents and patent applications cited herein.

In another embodiment of the invention, the picoplatin compositions of the invention used to prepare medicaments that are used in combination with an effective amount of a second medicament, such as an non-platinum containing anticancer agent. The latter agent can be co-administered to a patient in conjunction with administration of an embodiment of the present oral formulation

The anticancer drug can be a non-platinum based anticancer agent, or can be a platinum-based anticancer agent. Examples of a second anticancer agent or therapy comprising a molecular entity are provided above in Table 1, above. For example, a second anticancer agent can be a non-platinum based anticancer agent, or can be a platinum-based anticancer agent.

By “a non-platinum based anticancer agent” is meant a compound with anticancer and/or anti-cell proliferation activity that does not contain platinum, for example, a compound or drug can be selected from one of the following classes:

1. A compound of the camptothecin analogue class, i.e. any tumour cell growth inhibiting compound which is structurally related to camptothecin, and inhibits topoisomerase I; or a compound of the podophyllotoxin analogue class which inhibits topoisomerase II; or is a compound of the camptothecin analogue class which is an inhibitor of both topoisomerase I and II. Suitable compounds of the camptothecin analogue class include, but are not limited to, pure topoisomerase I inhibitors such as Topotecan, Irinotecan, 9-Aminocamptothecin, Rubitecan and Exatecan (DX-8951f); mixed topoisomerase I and topoisomerase II inhibitors such as XR-5000 and XR-11576; and suitable compounds of the podophyllotoxin analogue class which are pure topoisomerase II inhibitors include, but are not limited to, Etoposide and Teniposide. Such compounds also include, but are not limited to, any tumour cell growth inhibiting camptothecin analogue claimed or described in WO 93/09782 and the references cited therein (which are hereby incorporated herein by reference). The preparation of Topotecan (including pharmaceutically acceptable salts, hydrates and solvates thereof) as well as the preparation of oral and parenteral pharmaceutical compositions comprising topotecan and an inert, pharmaceutically acceptable carrier or diluent, is extensively described in U.S. Pat. No. 5,004,758 and European Patent Application Publication Number EP 0,321,122.
2. A taxane, such as Taxol (Paclitaxel) or Taxotere® (Docetaxel).
3. A growth-factor receptor inhibitor such as a growth factor receptor—protein-kinase inhibitor, including an epidermal growth factor receptor—class I tyrosine kinase inhibitor, for example, Iressa® (ZD1839 or Gefitinib) or Tarceva® (or Erlotinib)), and other inhibitors of growth factor function. Such growth factors include, for example, platelet derived growth factor, endothelial growth factor, vascular endothelial growth factor (VEGF), epidermal growth factor and hepatocyte growth factor and such inhibitors include growth factor antibodies and growth factor receptor antibodies, such as, e.g., Avastin® or Bevacizumab, and Erbitux® or Cetuximab, as well as serine/threonine kinase inhibitors. Also included are inhibitors of cell cycle kinases such as CDK-2, CDK-4 and CDK-6. Inhibitors of endothelial growth factor or vascular endothelial growth factor may act, at least in part, by inhibiting tumor angiogenesis.
4. An anti-metabolite such as 5-FU, S1, UFT, Capecitabine; a thymidylate synthase inhibitor such as Tomudex or ZD9331, or LY231514 (MTA, pemetrexed disodium) or Gemcitabine, or an antifolate such as Methotrexate.
5. A Vinca alkaloid such as Vinolrebine (Navelbine), Vincristine, Vinblastine or Vindesine.
6. An anti-angiogenic compound such as described in International Patent Application Publication Nos. WO 97/22596, WO 97/30035, WO 97/32856, WO 98/13354, WO 00/21955 and WO 00/47212.
7. An alkylating agent such as Melphalan, Cyclophosphamide, Ifosphamide or a nitroso-urea, such as Carmustine or Lomustine.
8. An Anthracyclin such as Doxrubicin, Epiribicin, Idarubicin, Amrubicin or Doxil®.
9. An anti-HER-neu compound, such as Herceptin (Trastuzumab).
10. A cytostatic agent such as an antioestrogen (for example, Tamoxifen, Toremifene, Raloxifene, Droloxifene, Iodoxyfene), a progestogen (for example, Megestrol Acetate), an aromatase inhibitor (for example, Anastrozole, Letrazole, Vorazole, Exemestane), an antiprogestogen, an antiandrogen (for example, Flutamide, Nilutamide, Bicalutamide, Cyproterone Acetate), LHRH agonists and antagonists (for example, Goserelin acetate, Luprolide), an inhibitor of testosterone 5α-dihydroreductase (for example, Finasteride) and an anti-invasion agent (for example, metalloproteinase inhibitors like Marimastat and inhibitors of urokinase plasminogen activator receptor function).
11. Antimitotics, natural and synthetic.
12. Interleukins and cytokines such as TNF.

### 13. Vaccines.

14. Uptake/efflux modulators such as mdr2.
15. Rescue agents.
16. Ca antagonists.

Potentiation agents, e.g., Leucovorin, that do not possess anti-cancer activity per se, can also be used in the present method.

A “platinum-based anticancer agent” can include other platinum agents, such as BBR3464, Satraplatin, Cisplatin, Carboplatin, Nedaplatin, Heptaplatin or Oxaliplatin, with a different mode of action or useful profile, may also be used with picoplatin.

These categories are provided as a summary of art-recognized classes of anti-cancer agents or other classes of active agent or adjuvant and not meant to be exclusive.

The second anticancer agent can be administered in an effective amount to the patient, concurrently with the oral picoplatin formulation, prior to administration of the oral picoplatin formulation, or subsequent to the oral picoplatin formulation, on a similar or diverse schedule of administration, provided that the second anticancer agent is administered at a dose, in a frequency, and for a duration of time sufficient to provide a beneficial effect to the patient when administered with the oral picoplatin formulation. The picoplatin oral formulation can be administered with (before, after or concurrently with) at least one platinum or non-platinum anticancer agent, which can be administered orally or parenterally. Preferably the picoplatin is administered concurrently (simultaneously or overlapping) or prior to the administration of the second anticancer agent. The second anticancer agent can be administered prior to the picoplatin. If it is a taxane it is preferably administered less than 10-20 hours to about 5 minutes prior to the picoplatin, e.g., about 1 hour to 15 minutes prior to the picoplatin.

Additive effects between the picoplatin and the additional anticancer agent can be observed, wherein the therapeutic effect of each agent is summed to provide a proportional increase in effectiveness. Synergistic effects between the picoplatin and the additional anticancer agent can be observed, wherein the combined effectiveness of the treatment is greater than the summed effectiveness of the two agents.

In various embodiments of the present invention the ionizing radiation employed may be X-radiation, γ-radiation, or β-radiation. The dosages of ionizing radiation will be those known for use in clinical radiotherapy. The radiation therapy used will include, for example, the use of γ-rays, X-rays, and/or the directed delivery of radiation from radioisotopes. Other forms of DNA damaging factors are also included in the present invention such as microwaves and UV-irradiation. It is most likely that all of these factors effect a broad range of damage to DNA, to the precursors of DNA, to the replication and repair of DNA, and to the assembly and maintenance of chromosomes. For example, X-rays may be dosed in daily doses of 1.8-2.0 Gy, 5 days per week for 5-6 weeks. Normally, a fractionaed dose will lie in the range 45-60 Gy. Single larger doses, for example 5-10 Gy, may be administered as part of a course of radiotherapy. Dosage ranges for radioisotopes vary widely, and depend upon the half-life of the isotope, the type and energy of the radiation emitted, and the rate of uptake by cells.

This application is related to Application No. PCT/US2008/008076, filed Jun. 27, 2008, entitled “Stabilized Picoplatin Dosage Form”; Application No. PCT/US2008/001746, filed Feb. 8, 2008, entitled “Encapsulated Picoplatin”; Application No. PCT/US2008/001752, filed Feb. 8, 2008, entitled “Stabilized Picoplatin Oral Dosage Form”; U.S. Ser. No. 10/276,503, filed Sep. 4, 2003, entitled “Combination Chemotherapy”; U.S. Ser. No. 11/982,841, filed Nov. 5, 2007, entitled “Use of Picoplatin to Treat Colorectal Cancer”; U.S. Ser. No. 11/935,979, filed Nov. 6, 2007, entitled “Use of Picoplatin to Treat Prostate Cancer”; U.S. Ser. No. 11/982,839, filed Nov. 5, 2007, entitled “Use of Picoplatin to Treat Small Cell Lung Cancer”; WO/98/045331, filed Apr. 3, 1998, entitled “Anti-VEGF Antibodies”; WO/96/040210, filed Jun. 7, 1996, entitled “Antibody and Antibody Fragments for Inhibiting the Growth of Tumors”; all of the above being incorporated by reference in their entireties herein.

This application is also related to U.S. Ser. No. 61/027,387, filed Feb. 8, 2008, entitled “Use of Picoplatin and Bevacizumab to Treat Colorectal Cancer”; U.S. Ser. No. 61/027,382, filed Feb. 8, 2008, entitled “Use of Picoplatin and Cetuximab to Treat Colorectal Cancer”; U.S. Ser. No. 61/027,360, filed Feb. 8, 2008, entitled “Picoplatin and Amrubicin to Treat Lung Cancer”; and U.S. Ser. No. 61/034,410, filed Mar. 6, 2008, entitled “Use of Picoplatin and Liposomal Doxorubicin Hydrochloride to Treat Ovarian Cancer”; all of the above being incorporated by reference in their entireties herein.

Furthermore, U.S. Pat. No. 7,060,808, issued Jun. 13, 2006, entitled “Humanized anti-EGF receptor monoclonal antibody”; and U.S. Pat. No. 4,673,668, issued Jun. 16, 1987, entitled “Aminonaphthacene derivatives”; are also incorporated herein by reference.

These patents and applications disclose, inter alia, useful agents for administration with picoplatin, methods of treatment, dosing regimens, and compositions.

### EXAMPLES

### Example 1

### HPLC Method for Picoplatin

### Conditions:

Column: Luna 5u C18(2) 250×4.6 mm
00G-4252-E0
(Phenomenex)

Mobile phase A: 0.2% TFA (v/v) in deionized water
(“di-water”)

Mobile phase B: Methanol HPLC grade

Flow rate: 1.0 mL/min

Detection wavelength: 267 nm

Column temperature: 35 deg C.

Sample temperature: 25 deg C.

Run time: 25 min

Sample diluent: Normal saline

TABLE I
Gradient

| Time (min) | % B |
|---|---|
| 0 | 5 |
| 4 | 5 |
| 13 | 35 |
| 14 | 100 |
| 18 | 100 |
| 19 | 5 |
| 25 | 5 |

### Example 2

### Determination of the Solubility of Picoplatin at Various pH Values

The objective of this study was to determine the solubility of picoplatin in aqueous solutions and to measure the effect of pH on picoplatin solubility.

TABLE II
pH Buffers

| Vial | pH | Buffer |
|---|---|---|
| 1 | 2 | 50 mM sodium phosphate |
| 2 | 3 | 50 mM sodium phosphate |
| 3 | 4 | 50 mM sodium acetate |
| 4 | 5 | 50 mM sodium acetate |
| 5 | 6 | 50 mM sodium citrate |
| 6 | 7 | 50 mM sodium phosphate |
| 7 | 8 | 50 mM sodium phosphate |
| 8 | 9 | 50 mM sodium bicarbonate |
| 9 | 10 | 50 mM sodium bicarbonate |
| 10 | Record | di-water |

### Procedure:

Picoplatin (10 mg) was weighed into 0.5 mL Eppendorf vials, for a total 10 vials, then 250 μL of buffer or water was added to the picoplatin. The vials were mixed for one minute. For each vial, the pH was measured. The vials were then placed on a shaker at 25 deg C. for 16 hr in dark and the pH was measured again. The solutions were filtered centrifugally through 0.45 uM Spin-X filters, then 50 mg of each filtrate was transferred into a respective HPLC vial. 1.5 mL of 0.9% NaCl solution (normal saline) was added to the HPLC vials, then HPLC analysis was performed immediately to determine the concentration of each sample.

TABLE III
pH of Picoplatin in Buffer Solutions

| | | Final pH | Solubility |
|---|---|---|---|
| Buffer | Initial pH | (filtrate) | (mg/mL)* |
| 50 mM sodium phosphate pH 2 | 1.83 | 2.05 | 0.74 |
| 50 mM sodium phosphate pH 3 | 3.51 | 3.82 | 0.98 |
| 50 mM sodium acetate pH 4 | 3.81 | 4.01 | 0.77 |
| 50 mM sodium acetate pH 5 | 4.88 | 4.97 | 0.84 |
| 50 mM sodium citrate pH 6 | 6.29 | 6.54 | 0.78 |
| 50 mM sodium phosphate pH 7 | 7.02 | 6.80 | 1.10 |
| 50 mM sodium phosphate pH 8 | 8.28 | 7.81 | 0.97 |
| 50 mM sodium bicarbonate pH 9 | 8.92 | 8.76 | 0.67 |
| 50 mM sodium bicarbonate pH 10 | 10.45 | 10.07 | 0.60 |
| Deionized H₂O | 5.24 | 4.66 | 1.23 |

*Assuming the density of the saturated solution is 1 g/mL

### Example 3

### Determination of the pH-Stability Profile of Picoplatin

The objective of this study was to determine the effects of pH on stability of picoplatin in aqueous solution and to assess the overall stability of picoplatin in an aqueous solution.

TABLE IV
pH Buffers

| Vial | pH | Buffer/Solvent |
|---|---|---|
| 1 | 2 | 50 mM sodium phosphate |
| 2 | 3 | 50 mM sodium phosphate |
| 3 | 4 | 50 mM sodium acetate |
| 4 | 5 | 50 mM sodium acetate |
| 5 | 6 | 50 mM sodium citrate |
| 6 | Record | di-water |
| 7 | Record | Normal Saline (NS) |

### Procedure:

Picoplatin (10 mg (+/−0.1 mg) was weighed into a 5 mL volumetric flask, then normal saline was added to the 5 mL volumetric mark and the sample mixed by inversion to dissolve all solid and obtain a 2 mg/mL stock solution. Then, to 1.125 mL buffer of specified pH or deionized water or normal saline in an HPLC vial was added 0.375 mL of the stock solution, which was mixed by vortex for 10 sec to obtain a 0.5 mg/mL test solution. Two vials were made up for each pH, which was checked.

The samples were then injected for HPLC analysis, analyzing each vial once in the following sequence: pH 6, pH 5, pH 4, pH 3, pH 2, deionized water, normal saline.

Then, one of each pair of vials for each solution was transferred to a 40° C. stability chamber, and the other to a 25° C. chamber.

The injection sequence was repeated after the elapse of 1 and 3 days, or until the samples were at least 20% degraded.

### Results:

The results are shown below in TABLES V-XIII

TABLE V
Picoplatin Recovery (% over initial) at 25 and
40° C. After 0, 1 and 2 Days

| | Initial | Initial | | Day |
|---|---|---|---|---|
| At 25° C. | pH | (T = 0) | Day 1 | 2 |
| pH 2, 50 mM sodium phosphate buffer | 2.78 | 100 | 99.79 | 94.85 |
| pH 3, 50 mM sodium phosphate buffer | 3.48 | 100 | 68.86 | 65.59 |
| pH 4, 50 mM sodium acetate buffer | 3.93 | 100 | 96.60 | 90.65 |
| pH 5, 50 mM sodium acetate buffer | 4.89 | 100 | 73.97 | 63.63 |
| pH 6, 50 mM sodium citrate buffer | 6.20 | 100 | 24.85 | 12.21 |
| Normal Saline | 5.54 | 100 | 102.67 | 97.45 |
| Deionized water | 5.59 | 100 | 29.87 | 25.43 |

* Based on peak area of picoplatin ONLY

TABLE VI
Picoplatin Recovery (% over initial) at 25 and
40° C. After 0, 1 and 2 Days

| | Initial | Initial | | |
|---|---|---|---|---|
| At 25° C. | pH | (T = 0) | Day 1 | Day 2 |
| pH 2, 50 mM sodium phosphate | 2.78 | 100 | 106.29 | 100.95 |
| buffer | | | | |
| pH 3, 50 mM sodium phosphate | 3.48 | 100 | 95.35 | 101.32 |
| buffer | | | | |
| pH 4, 50 mM sodium acetate buffer | 3.93 | 100 | 103.18 | 96.96 |
| pH 5, 50 mM sodium acetate buffer | 4.89 | 100 | 84.99 | 73.82 |
| pH 6, 50 mM sodium citrate buffer | 6.20 | 100 | 51.08 | 46.51 |
| Normal Saline | 5.54 | 100 | 113.71 | 109.15 |
| Deionized water | 5.59 | 100 | 100.59 | 94.68 |

* Based on combined peak area of picoplatin, Aquo 1 and Aquo 2.

TABLE VII
Picoplatin Recovery (% over initial) at 25 and
40° C. After 0, 1 and 2 Days

| | Initial | Initial | | |
|---|---|---|---|---|
| At 40° C. | pH | (T = 0) | Day 1 | Day 2 |
| pH 2, 50 mM sodium phosphate | 2.78 | 100 | 101.72 | 94.46 |
| buffer | | | | |
| pH 3, 50 mM sodium phosphate | 3.48 | 100 | 71.13 | 70.96 |
| buffer | | | | |
| pH 4, 50 mM sodium acetate buffer | 3.93 | 100 | 88.65 | 81.91 |
| pH 5, 50 mM sodium acetate buffer | 4.89 | 100 | 49.59 | 43.84 |
| pH 6, 50 mM sodium citrate buffer | 6.20 | 100 | 1.43 | 0.93 |
| Normal Saline | 5.54 | 100 | 103.79 | 102.59 |
| Deionized water | 5.59 | 100 | 29.58 | 31.41 |

* Based on peak area of picoplatin ONLY

TABLE VIII
Picoplatin Recovery (% over initial) at 25 and
40° C. After 0, 1 and 2 Days

| | Initial | Initial | | |
|---|---|---|---|---|
| At 40° C. | pH | (T = 0) | Day 1 | Day 2 |
| pH 2, 50 mM sodium phosphate | 2.78 | 100 | 108.39 | 100.68 |
| buffer | | | | |
| pH 3, 50 mM sodium phosphate | 3.48 | 100 | 113.45 | 167.66 |
| buffer | | | | |
| pH 4, 50 mM sodium acetate buffer | 3.93 | 100 | 95.58 | 88.04 |
| pH 5, 50 mM sodium acetate buffer | 4.89 | 100 | 62.23 | 53.01 |
| pH 6, 50 mM sodium citrate buffer | 6.20 | 100 | 28.32 | 38.77 |
| Normal Saline | 5.54 | 100 | 116.58 | 113.36 |
| Deionized water | 5.59 | 100 | 109.26 | 103.81 |

* Based on combined peak area of picoplatin, Aquo 1 and Aquo 2.

TABLE IX
Picoplatin purity (% over total peak area) at 25 and
40 deg C. after 0, 1 and 2 days

| | Initial | Initial | | Day |
|---|---|---|---|---|
| At 25° C. | pH | (T = 0) | Day 1 | 2 |
| pH 2, 50 mM sodium phosphate buffer | 2.78 | 97.9 | 90.2 | 88.0 |
| pH 3, 50 mM sodium phosphate buffer | 3.48 | 97.8 | 61.3 | 60.7 |
| pH 4, 50 mM sodium acetate buffer | 3.93 | 98.6 | 87.4 | 84.6 |
| pH 5, 50 mM sodium acetate buffer | 4.89 | 98.4 | 67.6 | 58.6 |
| pH 6, 50 mM sodium citrate buffer | 6.20 | 98.8 | 23.9 | 12.1 |
| Normal Saline | 5.54 | 95.3 | 90.4 | 89.2 |
| Deionized water | 5.59 | 98.9 | 29.5 | 25.6 |

* Based on peak area of picoplatin ONLY

TABLE X
Picoplatin purity (% over total peak area) at 25 and
40 deg C. after 0, 1 and 2 days

| | Initial | Initial | | Day |
|---|---|---|---|---|
| At 25° C. | pH | (T = 0) | Day 1 | 2 |
| pH 2, 50 mM sodium phosphate buffer | 2.78 | 97.9 | 97.0 | 94.7 |
| pH 3, 50 mM sodium phosphate buffer | 3.48 | 97.8 | 94.1 | 93.7 |
| pH 4, 50 mM sodium acetate buffer | 3.93 | 98.6 | 93.3 | 90.5 |
| pH 5, 50 mM sodium acetate buffer | 4.89 | 98.4 | 77.6 | 68.0 |
| pH 6, 50 mM sodium citrate buffer | 6.20 | 98.8 | 49.0 | 46.1 |
| Normal Saline | 5.54 | 95.3 | 100.0 | 100.0 |
| Deionized water | 5.59 | 98.9 | 98.1 | 95.4 |

* Based on combined peak area of picoplatin, Aquo 1 and Aquo 2.

TABLE XII
Picoplatin purity (% over total peak area) at 25 and
40 deg C. after 0, 1 and 2 days

| | Initial | Initial | | Day |
|---|---|---|---|---|
| At 40° C. | pH | (T = 0) | Day 1 | 2 |
| pH 2, 50 mM sodium phosphate buffer | 2.78 | 97.9 | 91.5 | 88.7 |
| pH 3, 50 mM sodium phosphate buffer | 3.48 | 97.8 | 57.4 | 58.0 |
| pH 4, 50 mM sodium acetate buffer | 3.93 | 98.6 | 77.8 | 74.7 |
| pH 5, 50 mM sodium acetate buffer | 4.89 | 98.4 | 44.6 | 41.0 |
| pH 6, 50 mM sodium citrate buffer | 6.20 | 98.8 | 2.0 | 0.9 |
| Normal Saline | 5.54 | 95.3 | 89.2 | 90.5 |
| Deionized water | 5.59 | 98.9 | 25.6 | 28.2 |

* Based on peak area of picoplatin ONLY

TABLE XIII
Picoplatin purity (% over total peak area) at 25 and
40 deg C. after 0, 1 and 2 days

| | Initial | Initial | | Day |
|---|---|---|---|---|
| At 40° C. | pH | (T = 0) | Day 1 | 2 |
| pH 2, 50 mM sodium phosphate buffer | 2.78 | 97.9 | 98.5 | 95.6 |
| pH 3, 50 mM sodium phosphate buffer | 3.48 | 97.8 | 91.5 | 114.3 |
| pH 4, 50 mM sodium acetate buffer | 3.93 | 98.6 | 83.5 | 80.3 |
| pH 5, 50 mM sodium acetate buffer | 4.89 | 98.4 | 55.9 | 49.6 |
| pH 6, 50 mM sodium citrate buffer | 6.20 | 98.8 | 38.6 | 37.1 |
| Normal Saline | 5.54 | 95.3 | 100.0 | 100.0 |
| Deionized water | 5.59 | 98.9 | 94.3 | 93.1 |

* Based on combined peak area of picoplatin, Aquo 1 and Aquo 2.

### Example 4

### Determination of Solubility of Picoplatin in Organic Solvents

The purpose of this study was to search for a solvent that can be used to facilitate dissolution of picoplatin into self-emulsifying vehicles.

### Solvent Selection Criteria:

Dissolve picoplatin to >20% w/w or 200 mg/mL

Volatile—removable by vacuum drying

Class 3 or injectable

Chemically compatible with picoplatin

TABLE XIV
Composition

| mg/g | F-1 | F-2 | F-3 | F-4 | F-5 | F-6 | F-7 |
|---|---|---|---|---|---|---|---|
| Acetonitrile | 180 | | | | | | |
| Tetrachloroethylene | | 180 | | | | | |
| Acetone | | | 180 | | | | |
| Methanol | | | | 180 | | | |
| THF | | | | | 180 | | |
| Isopropanol | | | | | | 180 | |
| Methylene chloride | | | | | | | 180 |
| Picoplatin | 20 | 20 | 20 | 20 | 20 | 20 | 20 |
| Total | 200 | 200 | 200 | 200 | 200 | 200 | 200 |

### Procedure:

Picoplatin (20+/−2 mg) was weighed into a series of 2 mL Eppendorf vials, 100 mg of each solvent was added respectively, then each sample was sonicated to mix and dissolve the picoplatin. If the picoplatin did not dissolve, additional aliquots of 100 mg solvent were added (to a maximum of 1.5 g), and the suspensions sonicated, until all of the solid did dissolve. Each sample was then dried on a Speedvac on low heat overnight to evaporate the solvent, then 200 mg deionized water was added to each vial. The supernatant (500 mg) was transferred from each vial into a respective HPLC vial, then 0.5 mL of the solvent used was added.

### Results:

The results are shown below in TABLE XV.

TABLE XV

| Solvent | Solubility (mg/g) |
|---|---|
| Acetonitrile | 1.30 |
| Tetrachloroethylene | 0.00 |
| Acetone | 0.14 |
| Methanol | 0.61 |
| THF | 1.81 |
| Isopropanol | 0.15 |
| Methylene chloride | 0.00 |
| DMSO | >200 (degradation) |
| N-methylpyrrolidone | >200 (peak shifted) |
| Benzyl benzoate | <5 |
| Benzyl alcohol | <5 |

### Example 5

### Determination of the Solubility of Picoplatin in Self-Emulsifying Vehicles

The purpose of this study was to find an oil: surfactant system(s) capable of dissolving Picoplatin to 10% w/w. The composition of the various samples is shown in TABLE XVI.

TABLE XVI
Composition

| mg/g | F-8 | F-9 | F-10 | F-11 | F-12 | F-13 | F-14 | F-15 | F-16 |
|---|---|---|---|---|---|---|---|---|---|
| Labrasol | 200 | | | | | | | | |
| Cremophor RH40 | | 200 | | | | | | | |
| Cremophor ELP | | | 200 | | | | | | |
| Gelucire 44/14 | | | | 200 | | | | | |
| Polysorbate 80, HP | | | | | 200 | | | | |
| Vitamin E TPGS | | | | | | 200 | 200 | 200 | 200 |
| PEG400 | 100 | 100 | 100 | 100 | 100 | 100 | 100 | 100 | 100 |
| Soy lecithin | 200 | 200 | 200 | 200 | 200 | 200 | | 200 | 200 |
| (high PC content) | | | | | | | | | |
| Soy lecithin | | | | | | | 200 | | |
| (low PC content) | | | | | | | | | |
| Medium chain | 300 | 300 | 300 | 300 | 300 | 300 | 300 | | |
| triglyceride | | | | | | | | | |
| Castor oil | | | | | | | | 300 | |
| Medium chain mono- | | | | | | | | | 300 |
| & di-glycerides | | | | | | | | | |
| Picoplatin | 200 | 200 | 200 | 200 | 200 | 200 | 200 | 200 | 200 |
| Total | 1000 | 1000 | 1000 | 1000 | 1000 | 1000 | 1000 | 1000 | 1000 |

### Procedure:

Picoplatin was weighed out to within +/−5% of the target weight, then solvent (e.g. DMSO USP) was added to dissolve. Then, oil, lecithin, PEG400 and a surfactant were mixed to within +/−5-10% of the target weight, then ethanol was added to homogeneity. The two solutions were combined, then vacuum dried until the residual solvent was less than 1% of the dry weight. The dry formulation was examined under a microscope for crystals. If crystals were present, the sample was centrifuged to the pellet the crystals. Then 10 mg of the supernatant was removed and 5 g normal saline added. The drug concentration was analyzed by HPLC.

TABLE XVII

| Result | F-8 | F-9 | F-10 | F-11 | F-12 | F-13 | F-14 | F-15 | F-16 |
|---|---|---|---|---|---|---|---|---|---|
| Appearance of the dry | Free of crystalline particles | | | | | | | | |
| formulations | | | | | | | | | |
| Form | Liquid | Liquid | Liquid | Semi-solid | Liquid | Semi-solid | Semi-solid | Semi-solid | Liquid |
| pH | 4.28 | 5.18 | 5.5 | 4.83 | 5.86 | 5.67 | 4.96 | 4.67 | 5.23 |
| Picoplatin Conc. | 0.67 | 0.42 | 0.19 | 0.19 | 0.36 | 0.33 | 0.18 | 3.99 | 0.40 |
| (% w/w) | | | | | | | | | |
| Picoplatin Purity | 42.4 | 6.2 | 7.3 | 6.3 | 11.5 | 7.3 | 6.6 | 73.2 | 19.8 |
| (Area %) | | | | | | | | | |

### Example 6

### Degradation of Picoplatin in DMSO and pH Buffers at 25° C.

The purpose of this study was to obtain a profile of picoplatin in DMSO and pH buffers or water.

TABLE XVIII
Materials

| mg/mg | F-28 | F-29 | F-30 | F-31 | F-32 | F-33 | F-34 |
|---|---|---|---|---|---|---|---|
| Picoplatin | 0.5 | 0.5 | 0.5 | 0.5 | 0.5 | 0.5 | 0.5 |
| DMSO | 950 | 950 | 950 | 950 | 950 | 950 | 500 |
| glacial acetic acid | 50 | | | | | | |
| Normal saline | | 50 | | | | | 500 |
| pH 2 buffer | | | 50 | | | | |
| pH 4 buffer | | | | 50 | | | |
| pH 6 buffer | | | | | 50 | | |
| Di-water | | | | | | 50 | |
| Total | 1000 | 1000 | 1000 | 1000 | 1000 | 1000 | 1000 |

### Procedure:

Picoplatin (0.5 mg+/−0.01) was weighed out into a 1.5 mL HPLC vials for a total of 7 vials. DMSO and the 2^{nd} solvent were weighed out in a separate 2 mL Eppendorf vial and mixed well. Then, 1 mL of the DMSO mixture with solvent was transferred into the HPLC vial containing picoplatin, then mixed by vortex for 10 sec to make sure all solid was dissolved.

The samples were then analyzed by HPLC, running the sequence 4-5 times, or until at least 20% of the picoplatin had degraded

### Example 7

### Preparation of Picoplatin Nanoparticles

The purpose of this study was to generate nanometer sized and preferably non-crystalline particles of picoplatin.

TABLE XIX

| Compound | % w/w |
|---|---|
| Picoplatin | 2.5 |
| Soy lecithin | 5 |
| deionized water | 92.5 |
| Total | 100 |

### Procedure:

Soy lecithin and deionized water were weighed out, then mixed with a high-shear mixer to obtain a uniform dispersion. Picoplatin was added and mixed well, the suspension being microfluidized until the particle size reached a minimum by laser light scattering or disappearance of crystalline particles. Then, the nanosuspension was freeze-dried to obtain a dry powder.

### Results:

The results are shown below in TABLE XX.

TABLE XX

| | Particle size | | Purity by | Crystalline |
|---|---|---|---|---|
| In-process sample | by LLS | | HPLC | particles |
| Pre-microfluidization | 10-20 | micron | 94.4% | A lot |
| Post-microfluidization | 643 | nm | 93.8% | Not seen |
| Post-lyophilization | 584 | nm | 95.1% | A few |

A significant size reduction from about 10 to 0.5 micron in diameter corresponding to about 400-fold increase in particle surface area was obtained by microfluidization. It was found that picoplatin retains its integrity (purity) after the microfluidization and lyophilization process. Also, a reduction in crystallinity was apparent.

### Example 8

### Determination of Picoplatin Stability in NMP

The purpose of this study was to develop a profile of picoplatin in N-methyl-pyrrolidone at 25° C. and at 5° C.

TABLE XXI
Composition

| mg/g | F-45 | F-46 | F-47 | F-48 | F-49 |
|---|---|---|---|---|---|
| Picoplatin | 0.5 | 0.5 | 0.5 | 0.5 | 0.5 |
| NMP | 1000 | 800 | 500 | 200 | |
| NS | | 200 | 500 | 800 | 1000 |
| Total | 1000.5 | 1000.5 | 1000.5 | 1000.5 | 1000.5 |

TABLE XXII
Composition

| mg/g | F-45 | F-46 | F-47 | F-48 | F-49 |
|---|---|---|---|---|---|
| Stock | 200 | 200 | 200 | 200 | HPLC Std |
| NMP | 800 | 600 | 300 | | |
| NS | | 200 | 500 | 800 | |
| Total | 1000 | 1000 | 1000 | 1000 | |

### Procedure:

In a 2 mL Eppendorf vial, 2.000 mg picoplatin was weighed out, 800 mg NMP added, and the mixture vortexed to dissolve picoplatin to obtain a stock solution, of which 200 mg was transferred into Eppendorf vials for total of 4 vials. An appropriate amount of normal saline was added and mixed well by vortex for approximately 10 seconds, then 500 mg was transferred into an HPLC vial, and an HPLC analysis run. Then, the remainder of the solution was dried in a lyophilizer until all the liquid was gone and 500 mg normal saline was added to each vial and mixed well by vortex for 20 seconds, transferred 500 mg into an HPLC vial. Ran HPLC with a 0.5 mg/mL standard.

### Results:

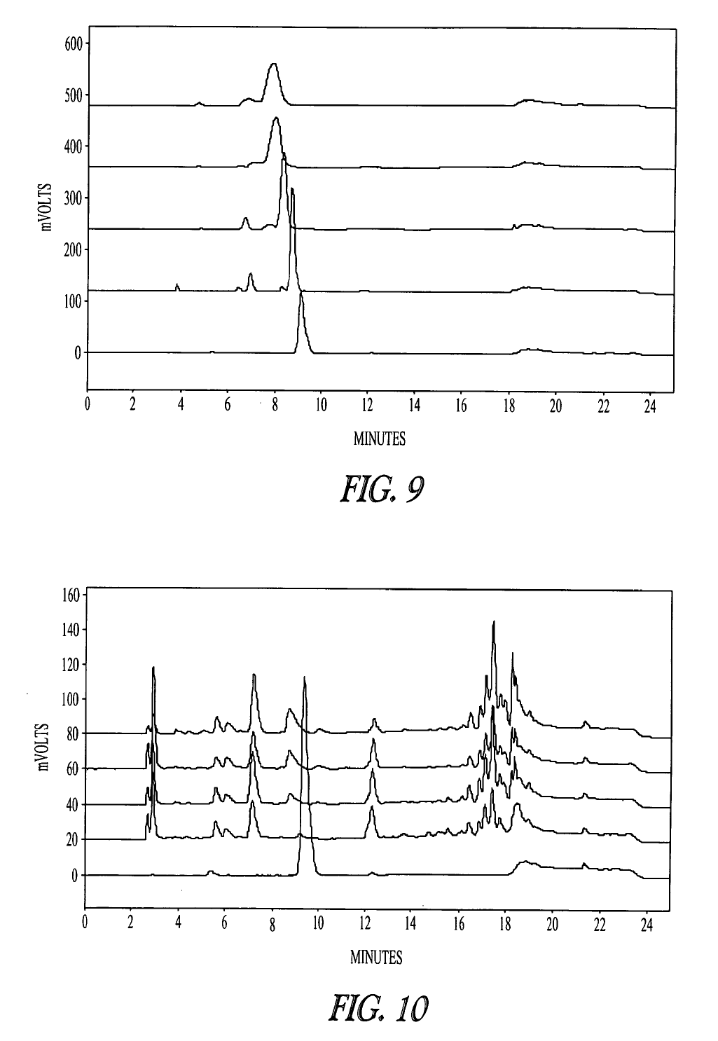

Representative HPLC chromatograms are shown in FIGS. 9 and 10.

### Example 9

### Optimization of Picoplatin Nanoparticle Formulations

The purpose of this study was to prepare and compare stability of nanoparticles using various stabilizers by microfluidization.

TABLE XXIII
Composition (% w/w)

| | | | | | | | |
|---|---|---|---|---|---|---|---|
| Compound | F-37 | F-38 | F-39 | F-40 | F-41 | F-42 | F-43 |

Composition (% w/w)

| | | | | | | | |
|---|---|---|---|---|---|---|---|
| Picoplatin | 2.5 | 2.5 | 2.5 | 2.5 | 2.5 | 2.5 | 2.5 |
| Soy lecithin S-45 | 5 | | | | | | |
| Soy lecithin S-75 | | 5 | | | | | |
| Soy lecithin PL-90 | | | 5 | | | | |
| Soy lecithin PL-90H | | | | 5 | | | |
| Vitamin E Succinate, pH7 | | | | | 97.5 | | |
| 5% pre-made dispersion in | | | | | | | |
| NS | | | | | | | |
| Oleic acid (Croda), pH 7, 5% | | | | | | 97.5 | |
| pre-made dispersion in water | | | | | | | |
| Sodiumcaseinate, pH 7, 5% | | | | | | | 97.5 |
| pre-made dispersion in water | | | | | | | |
| Di-water | 92.5 | 92.5 | 92.5 | 92.5 | | | |
| Total | 100 | 100 | 100 | 100 | 100 | 100 | 100 |

Composition (mg/10 g)

| | | | | | | | |
|---|---|---|---|---|---|---|---|
| Picoplatin | 25 | 25 | 25 | 25 | 25 | 25 | 25 |
| S-45 | 50 | 0 | 0 | 0 | 0 | 0 | 0 |
| S-75 | 0 | 50 | 0 | 0 | 0 | 0 | 0 |
| PL-90 | 0 | 0 | 50 | 0 | 0 | 0 | 0 |
| PL-90H | 0 | 0 | 0 | 50 | 0 | 0 | 0 |
| VES pH7 5% pre-made | 0 | 0 | 0 | 0 | 975 | 0 | 0 |
| dispersion in water | | | | | | | |
| Oleic acid (Croda), pH 7, 5% | 0 | 0 | 0 | 0 | 0 | 975 | 0 |
| pre-made dispersion in water | | | | | | | |
| Sodium caseinate, pH 7, 5% | 0 | 0 | 0 | 0 | 0 | 0 | 975 |
| pre-made dispersion in water | | | | | | | |
| Di-water | 925 | 925 | 925 | 925 | 0 | 0 | 0 |
| Total | 1000 | 1000 | 1000 | 1000 | 1000 | 1000 | 1000 |

*Added additional 10 g of di-water to each.

### Procedure

Lecithin PL, picoplatin and deionized water were weighed out into a 50 mL falcon tube and mixed by high-shear mixer at 8000 RPM for 2 minutes until all of the solid was uniformly dispersed. A micro fluidizer with a Z-chamber was set up and the sample was processed for about 1100 strokes. 1 g each was transferred into 3 mL glass vial for a total of ˜15 vials, which were freeze-dried to obtain a “lyophilizate”.

One vial of the lyophilizate was reconstituted by adding di-water and mixing well to form a suspension. “Post-lyo”

For all samples, the following tests were performed at (T=0):

Micrograph at 200×, laser light scattering (LLS),

HPLC (dilute to 0.5 mg/mL with NS) for post lyophilization sample only

### Results

The results are shown below in TABLE XXIV.

TABLE XXIV
Results

| | F-37 | F-38 | F-39 | F-40 | F-41 | F-42 | F-43 |
|---|---|---|---|---|---|---|---|
| Microscopic examination after 1100 passes | + | ++ | ++ | + | +++ | +++ | + |
| Microscopic examination after keeping the | ++ | Not | Not | +++ | Not | Not | + |
| suspension at 5° C. for 24 hr | | tested | tested | | tested | tested | |
| Microscopic examination after keeping the | ++ | Not | Not | +++ | Not | Not | + |
| suspension at 5° C. for 72 hr | | tested | tested | | tested | tested | |
| Microscopic examination after keeping the | ++ | Not | Not | +++ | Not | Not | + |
| suspension at 25° C. for 24 hr | | tested | tested | | tested | tested | |
| Microscopic examination after keeping the | ++ | Not | Not | +++ | Not | Not | + |
| suspension at 25° C. for 72 hr | | tested | tested | | tested | tested | |
| Microscopic examination after reconstitution | ++ | Not | Not | ++ | Not | Not | + |
| of the lyophile | | tested | tested | | tested | tested | |
| Microscopic examination after reconstitution | +++ | Not | Not | +++ | Not | Not | ++ |
| of the lyophile at 25° C. for 72 hr | | tested | tested | | tested | tested | Protein |
| | | | | | | | precipitate |

+++ A large number of visible crystals in 1-5 micron
++ Some crystalline particles
+ Few crystalline particles

TABLE XXV
Particle diameter by Laser Light Scattering (LLS) in nm

| | Sample ID | | |
|---|---|---|---|
| | F-37 | F-40 | F-43 |
| After 1100 passes | 746 | 3386 | 136 |
| After keeping the suspension | 844 | 1630 | 188 |
| at 5° C. for 24 hr | | | |
| After keeping the suspension | 1406 | 758 | 228 |
| at 25° C. for 24 hr | | | |
| Reconstituted suspension | 1126 | 1740 | 1104 |
| at 25° C. for 72 hr | | | |

TABLE XXVI
HPLC analysis for picoplatin in nanoparticles

| | Concentration | |
|---|---|---|
| Sample ID | (mg/mL)* | Purity |
| F37 reconstituted suspension (fresh) | 0.36 | 97.3 |
| F40 reconstituted suspension (fresh) | 0.48 | 94.8 |
| F43 reconstituted suspension (fresh) | 0.59 | 94.5 |
| Based picoplatin peak only | | |
| F37 reconstituted suspension (fresh) | 0.36 | 100.0 |
| F40 reconstituted suspension (fresh) | 0.50 | 100.0 |
| F43 reconstituted suspension (fresh) | 0.60 | 97.7 |

Based on combination of pico, Aquo 1 and Aquo 2 peaks
*The target concentration is 0.5 mg/mL

### Example 10

### Preparation of a Second Batch of Picoplatin Nanoparticles in 5% Sod. Caseinate Dispersion.

The purpose of this study was to reproduce the results from the previous experiment and to try using a rotary evaporator to remove water.

TABLE XXVII
Composition:

| | |
|---|---|
| Compound | F-50 |

Composition (% w/w)

| | |
|---|---|
| Picoplatin | 1.25 |
| Sodiumcaseinate, pH 7, 5% | 2.5 |
| pre-made dispersion in water | |
| Di-water | QS |
| Total | 100 |

Composition (mg/40 g)

| | |
|---|---|
| Picoplatin | 500 |
| Sodiumcaseinate, pH 7, 5% | 19500 |
| pre-made dispersion in water | |
| Di-water | 20000 |
| Total | 40000 |

### Procedure

100 g of a 5% sodium caseinate dispersion and 100 g of deionized water were weighed into an Erlenmeyer flask, and the pH adjusted to 6 using HCl/NaOH. The solution was sparged with Nitrogen gas for 10 minutes, then 39.5 g of the dispersion transferred into a 100 mL Erlenmeyer flask. 500 mg picoplatin was added and mixed under high shear conditions at 8000 RPM for 5 minutes. A 500 mg sample was processed in a microfluidizer with a Z-chamber for 2200 strokes and the pH recorded. The remainder of the sample was dried at 40° C. on a rotary evaporator for 2 hr, then vacuum dried at 25° C. and 150 mTorr for 16 hr. The residue was ground into a fine powder, then the moisture content determined by TG/DTA along with a picoplatin standard. A moisture uptake study was performed by placing 10 mg in 3 HPLC vials and keeping them at 25 deg C./60% RH, 30 deg C./65% RH and 40 deg C./75% RH respectively, exposed overnight. An HPLC analysis and a microscopic examination were performed.

### Results

FIG. 11 shows a thermogravimetric/differential thermal analysis (TG/DTA) scan of micronized picoplatin powder.

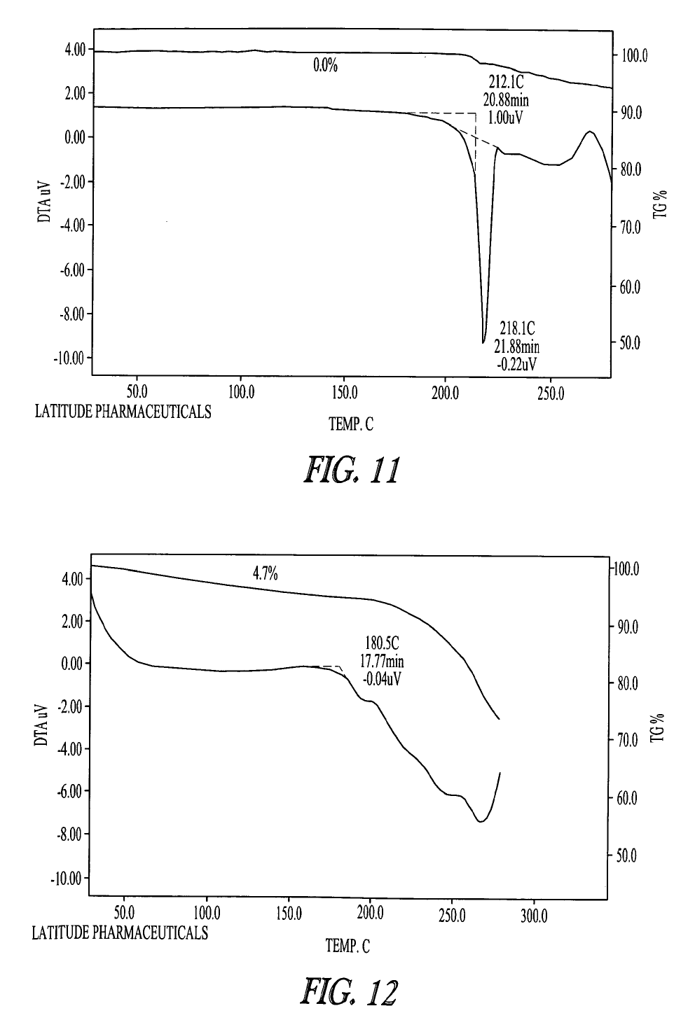

FIG. 12 shows a thermogravimetric/differential thermal analysis (TG/DTA) scan of TG/DTA of F50 Picoplatin nanoparticles in sodium caseinate.

Particle size in the reconstituted suspension could not be measured due to presence of large non-crystalline caseinate agglomerates, which interfered with the laser light scattering measurement. However, microscopic examination revealed that there was few crystalline particles in the micron size range, indicating that picoplatin remained in nanometer size (possibly less than 300-400 nm).

TABLE XXVIII
Hygroscopicity Data

| Temperature/Humidity Conditions | % Weight Gain |
|---|---|
| 25° C./60% RH overnight | 0.36 |
| 30° C./65% RH overnight | 0.73 |
| 40° C./75% RH overnight | 3.6 |

TABLE XXIX
HPLC Results

| Lot: 69-1-68 | Assay (mg/g)* | % Peak area |
|---|---|---|
| Picoplatin | 569.0 | 93.9 |
| Aqua 1 | 761.7 | 1.7 |
| Aqua 2 | 848.4 | 1.15 |
| Total | 573.7 | 96.75 |

*Theoretical assay value = 333.3 mg/g or 33.3% w/w. The higher-than-theoretical assay value may be due to presence of volatile components (e.g. water) in the sodium caseinate starting material.

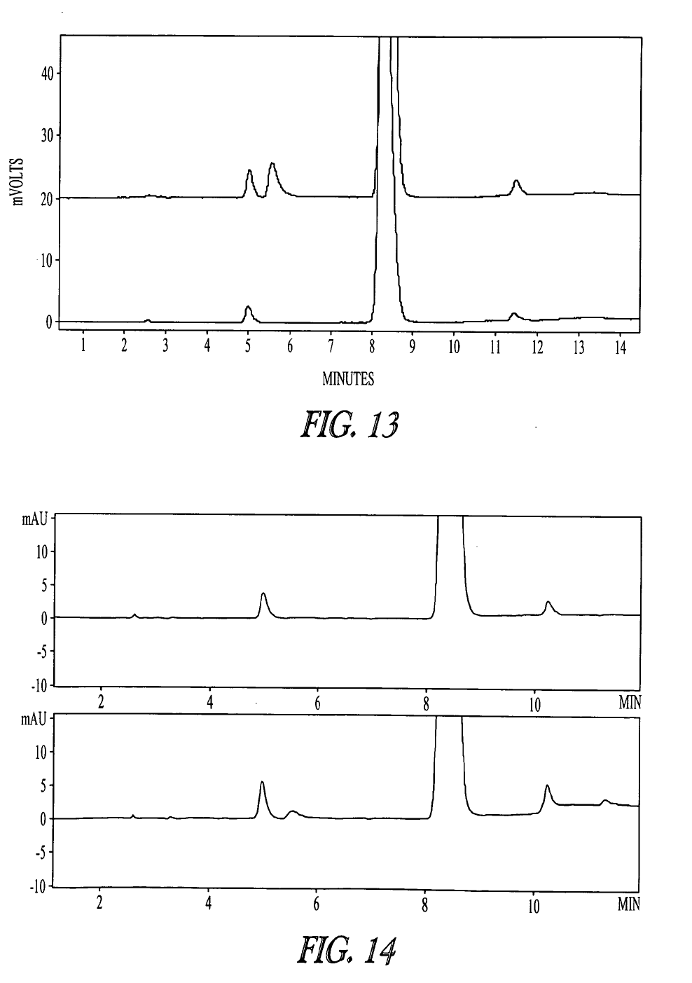

FIG. 13 shows a representative HPLC chromatogram of picoplatin nanoparticles. From the top down: 0.5 mg/mL picoplatin nanoparticles in normal saline and 0.5 mg/mL picoplatin standard in normal saline. One unknown peak at 5.5 min (not Aquo #1).

### Example 11

### Solid Dispersion of Picoplatin Using Hot Melt Method

The purpose of this study was to determine if it is possible to dissolve picoplatin in a molten solution of a solid matrix excipient without decomposition of picoplatin. The second purpose of this study is to verify the solid matrix form for crystallinity by DSC.

TABLE XXX
Composition (mg)

| Component, grade | MP | F-51 | F-52 | F-53 | F-54 | F-57 |
|---|---|---|---|---|---|---|
| Gelucire 50/13 | 45 | 950 | | | | |
| poloxamer 188 | 52 | | 950 | | | |
| PEG 8000 | 60 | | | 950 | | |
| Sorbitan monostearate | 57 | | | | 950 | |
| (SPAN 60) | | | | | | |
| Kollidon K-90 | 150 | | | | | 950 |
| Picoplatin | | 50 | 50 | 50 | 50 | 50 |

### Procedure:

The selected excipient was weighed out into a 3 mL glass vial, then warmed up to a temperature of about 5-10° C. above the melting point of the matrix material using a hot plate. Picoplatin was added and the mixture stirred at about 100° C. for 1 hr, or for the sorbitan monostearate sample, at about 150° C. The samples were then cooled quickly on a chilled metal block.

### Observations:

Picoplatin dissolved in molten Gelucire 50/13 and in SPAN 60, but not in PEG, poloxamer or Kollidon, suggesting picoplatin is more soluble in lipids. The Gelucire 50/13 picoplatin mixture appeared to contain intact picoplatin, but the SPAN 60 picoplatin mixture turned brown on heating

### Example 12-1

### Solid Dispersion of Picoplatin Using Hot Melt Method

The purpose of this study is to determine the solubility of picoplatin in Gelucire 50/13 and to try two more low MP lipids

TABLE XXXI
Composition (mg)

| Component, | | | | | | | | | |
|---|---|---|---|---|---|---|---|---|---|
| supplier, grade | MP | F-59 | F-60 | F-61 | F-62 | F-63 | F-64 | F-65 | F-66 |
| Gelucire 50/13 | 45 | 90 | 80 | 70 | 60 | 50 | 40 | | |
| Gelucire 44/14 | 44 | | | | | | | 70 | |
| Vitamin E TPGS | 40 | | | | | | | | 70 |
| Picoplatin | | 10 | 20 | 30 | 40 | 50 | 60 | 30 | 30 |

### Procedure

The selected excipient and the picoplatin (+/−2 mg) were weighed into a HPLC glass vial, and vortexed to mix. The mixture was heated to 60° C. to form a complete melt, and stirred and observed to determine if complete dissolution of the picoplatin occurred. The sample was heated at 60 deg C. for 1 hour for F-59 to F-66, and F-61 to F-66 received additional 30 min heating at 80 deg C. The samples were then cooled immediately by placing the vial in a chilled metal block.

### Example 12-2

### Solubility of Picoplatin in Gelucire 50/13

The purpose of this study was to determine the solubility of picoplatin in Gelucire 50/13 at less than 10% and to test one more lipid (Compritol 888 ATO) at 5%

TABLE XXXII
Composition (mg)

| Component, supplier, | | | | | | |
|---|---|---|---|---|---|---|
| grade | MP | F-67 | F-68 | F-69 | F-70 | F-71 |
| Gelucire 50/13 | 45 | 95 | 94 | 93 | 92 | |
| Compritol 888 ATO | 70 | | | | | 95 |
| Pico | | 5 | 6 | 7 | 8 | 5 |

### Procedure:

The lipid and picoplatin (+/−2 mg) were weighed into a HPLC glass vial, then vortexed to mix. Then, a glass beaker with Miglyol oil and placed it on a hot plate set to 100° C. All mixtures were heated for 2 hours (100 deg C.) and vortexed from time to time. After heating, all samples were cooled rapidly by placing the vial in a chilled metal block.

### Observations:

All turned clear. The solutions of F-67 and F-68 appeared slightly clearer than the others. The results of Samples F-51 to F-71 are shown below in Tables XXXIII and XXXIV.

TABLE XXXIII
Results of Examples 12-1 and 12-2

| Matrix | F-51 | F-52 | F-53 | F-54 | F-57 | F-59 | F-60 | F-61 | F-62 |
|---|---|---|---|---|---|---|---|---|---|
| Gelucire 50/13 | YTO* | | | | | YTO | YTO | YQC | YQC |
| Poloxamer 188 | | YQC | | | | | | | |
| PEG 8000 | | | YQC | | | | | | |
| SPAN 60 | | | | BTO* | | | | | |
| Kollidon K-90 | | | | | N | | | | |
| Gelucire 44/14 | | | | | | | | | |
| Vitamin E TPGS | | | | | | | | | |
| Compritol 888 ATO | | | | | | | | | |

Characterization of picoplatin-matrix mixture upon heating
Key:
Y= yellow
B= brown
O= oily
C= creamy
T= translucent
Q= opaque
N= never melted
*= No DSC peak

TABLE XXXIV
Results of Examples 12-1 and 12-2

| Matrix | F-63 | F-64 | F-65 | F-66 | F-67 | F-68 | F-69 | F-70 | F-71 |
|---|---|---|---|---|---|---|---|---|---|
| Gelucire 50/13 | YQC | YQC | | | YTO* | YTO | YTO | YTO | |
| Poloxamer 188 | | | | | | | | | |
| PEG 8000 | | | | | | | | | |
| SPAN 60 | | | | | | | | | |
| Kollidon K-90 | | | | | | | | | |
| Gelucire 44/14 | | | YTO | | | | | | |
| Vitamin E TPGS | | | | YTO | | | | | |
| Compritol 888 ATO | | | | | | | | | YTO |

Characterization of picoplatin-matrix mixture upon heating
Key:
Y= yellow
B= brown
O= oily
C= creamy
T= translucent
Q= opaque
N= never melted
*= No DSC peak

TABLE XXXV
Concentration of Picoplatin in
Gelucire 50/15 hot melt (F-51, 5% load)

| | Concentration | Purity |
|---|---|---|
| | (mg/g)* | (% peak area) |
| 5% picoplatin in | 68.7 | 96.95 |
| Gelucire 50/15 | | |

*Theoretical concentration is 50 mg/g (5% w/w)

FIG. 14 shows a representative HPLC trace of picoplatin in Gelucire 50/15.

FIG. 15 shows a representative DSC for Picoplatin in Gelucire 50/15 hot melt. From top down: Gelucire 50/15, 5% picoplatin in Gelucire 50/15 hot melt, and picoplatin API.

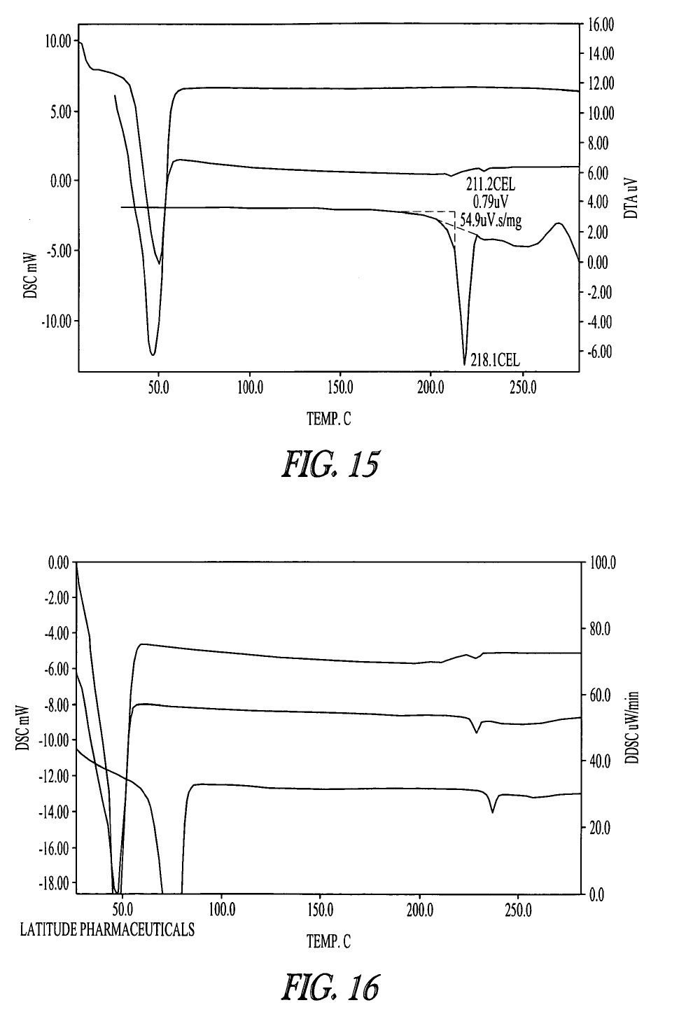

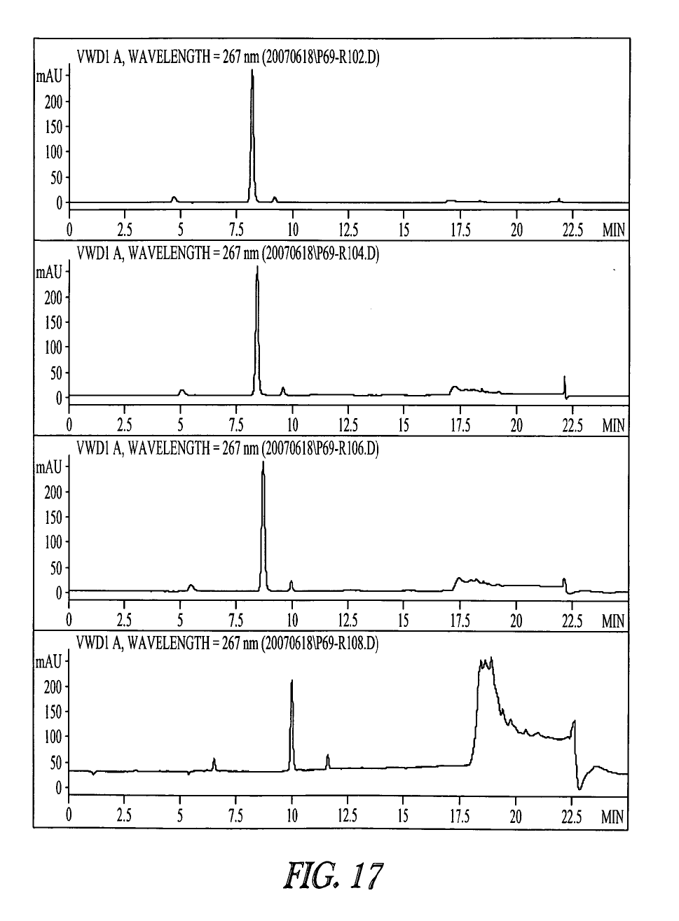

FIG. 16 shows a representative DSC for Picoplatin in hot melt. From top down: 5% picoplatin in Gelucire 50/15, 6% picoplatin in Gelucire 50/15 and 5% in Compritol 888 ATO.

TABLE XXXVI
Heat of Fusion for 5% picoplatin in Gelucire 50/15, 6% picoplatin
in Gelucire 50/15 and 5% picoplatin in Compritol 888 ATO.

| | Heat of Fusion (mJ/mg) |
|---|---|
| | for the endothermic |
| Sample | peak at 220-250° C. |
| Picoplatin API | 54.9 |
| F51- 5% picoplatin in Gelucire 50/15 | 10.0 |
| F68- 6% picoplatin in Gelucire 50/15 | 31.7 |
| F71- 5% picoplatin in Compritol 888 ATO | 36.3 |

### Example 13

### Preparation of 50% W/W Picoplatin Suspension in Medium Chain Triglyceride (MCT) Oil

### Objective:

To prepare 50% w/w picoplatin nano-suspension in MCT oil

TABLE XXXVII
Materials

| | | | |
|---|---|---|---|
| Compound | F-73 | F-74 | F-75 |

Composition (% w/w)

| | | | |
|---|---|---|---|
| Picoplatin | 5 | 5 | 5 |
| Miglyol 812 | 95 | 90 | 90 |
| Phospholipon 90G | | 5 | |
| Polysorbate 80 | | | 5 |
| Total | 100 | 100 | 100 |

Composition (g/30 g)

| | | | |
|---|---|---|---|
| Picoplatin | 1.5 | 1.5 | 1.5 |
| Miglyol 812 | 28.5 | 27 | 27 |
| Phospholipon 90G | | 1.5 | 1.5 |
| Polysorbate 80 | | | |
| Total | 30 | 30 | 30 |

### Procedure:

Picoplatin was weighted out into a 50 mL Falcon tube, MCT oil was added to the tube (final picoplatin concentration was 5% w/w). PL-90 or Polysorbate 80 was then added, and mixed using a high shear mixer (IKA @ 5 setting for 3 minutes), then microfluidized using M110EH at 25000 psi and a Z-chamber to obtain submicron particles. Chill the chamber with ice. Maintain the suspension during processing at below 40-50 deg C.

Samples were removed and average size determined by laser light scattering. Allow the suspension settle down and remove supernatant to obtain about 50% w/w suspension. Store at 2-8° C. Observe under microscope and measure size at T-0 and Day-1. Run HPLC (diluted in normal saline to 0.5 mg/mL) at Day-7

### Results

TABLE XXXVIII
Process and Size

| | F-73 | F-74 | F-75 |
|---|---|---|---|
| Passes | 200 | 200 | 200 |
| Size at T0 | 430 nm | 482.33 | 807 nm |
| Size at Day 1 | 638 nm | 602.7 nm | 576 nm |
| Size at Day 7 | 474 nm | 485 nm | 186 nm |
| Observation under | Aggregated | Uniformly | Aggregated |
| microscope | particles | separated particles | particles and |
| | | | phase separated |

TABLE XXXIX
HPLC (Method #1)

| | | Std | | | |
|---|---|---|---|---|---|
| Peak Area | RT | (0.5 mg/mL | | | |
| (% of total) | (min) | in NS) | F-73 | F-74 | F-75 |
| Picoplatin | | 91.7 | 90.18 | 88.98 | 80.13 |
| Aqua 1 | 4.6 | 4.9 | 5.59 | 6.12 | 9.81 |
| Aqua 2 | 9.09 | 3.4 | 4.23 | 4.70 | 9.62 |
| Unk#1 | 5.7 | 0 | 0 | 0.24 | 0 |
| Unk#2 | 6.2 | 0 | 0 | 0 | 0.88 |
| Total | | 100.0 | 100.01 | 100.03 | 100.43 |

*Oil phase (supernatent) contained no picoplatin, as determined by HPLC.

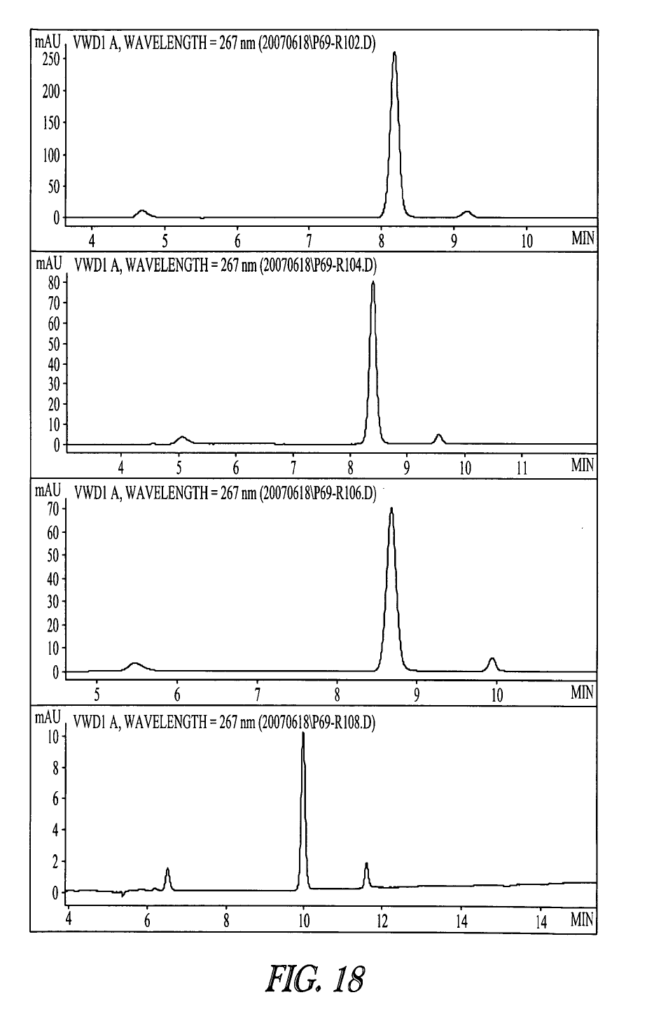

FIG. 17 shows HPLC traces, from the top down: 0.5 mg/mL standard in normal saline, F73-picoplatin in MCT, F74-picoplatin in MCT and PL90G, and F75-picoplatin in MCT and Polysorbate 80.

FIG. 18 shows zoomed-in views of the HPLC traces of FIG. 17 From the top down: 0.5 mg/mL standard in NS, F73-picoplatin in MCT, F74-picoplatin in MCT and PL90G, and F75-picoplatin in MCT and Polysorbate 80.

### Example 14

### Preparation of 50% W/W Picoplatin Suspension in MCT and Oils

### Objective:

% To prepare final concentration of 50% w/w picoplatin suspension in oils. To compare microfluidization efficiency in oils with different viscosity

TABLE XL
Materials

| | | | | |
|---|---|---|---|---|
| Compound | F-76 | F-77 | F-78 | F-79 |

Composition (% w/w)

| | | | | |
|---|---|---|---|---|
| Picoplatin | 10 | 10 | 10 | 10 |
| Miglyol MCT | 85 | | | |
| Ethly oleate | | 85 | | |
| Capmul MCM | | | 85 | |
| Soybean oil, super refined | | | | 85 |
| PL-90 | 5 | 5 | 5 | 5 |
| Normal saline | 10 | | | |
| Total | 110 | 100 | 100 | 100 |

Composition (g/tube)

| | | | | |
|---|---|---|---|---|
| Picoplatin | 3 | 3 | 3 | 3 |
| Miglyol MCT | 25.5 | 0 | 0 | 0 |
| Ethyl oleate | 0 | 25.5 | 0 | 0 |
| Capmul MCM | 0 | 0 | 25.5 | 0 |
| Soybean oil, super refined | 0 | 0 | 0 | 25.5 |
| PL-90 | 1.5 | 1.5 | 1.5 | 1.5 |
| Normal saline | 3 | 0 | 0 | 0 |
| Total | 33 | 30 | 30 | 30 |

### Procedure:

Weigh out Picoplatin into a 50 mL Falcon tube. Record weight. Add oil and PL90. Record weight. Mix using a high shear mixer, IKA @ 5 setting for 3 minutes

Microfluidize using Z-chamber for 200 passes. Record the pass# and final particle size. Let the sample settle down and remove 90% of sample weight of supernatant to obtain 50% w/w suspension. HPLC for purity. Store at 2-8° C.

### Results:

F76 formed large aggregates and was not able to be microfludized. However, small amount of sample with additional amount of PL90 added (double amount) was tested and it appeared to have smaller particle size and possibly can be microfludized. It will be tested in the next study.

F79 formed large aggregates and was not able to be microfludized.

F78 became a waxy semi-solid and therefore, could not be processed by either high-shear or microfluidization.

F77 was the only formulation that could be microfludized. The particle size after microfluidization for 200 passes is 919 nm by LLS.

TABLE XLI
Purity % by HPLC (Method #1) for F77

| Peak Area | RT | Std | |
|---|---|---|---|
| (% of total) | (min) | (0.5 mg/mL in NS) | F-77 |
| Picoplatin | 7.6 | 91.9 | 77.1 |
| Aqua 1 | 4.7 | 3.5 | 10.6 |
| Aqua 2 | 10.3 | 4.4 | 10.8 |
| Unk#1 | 4.3 | 0.2 | 1.5 |
| Total | | 100.0 | 100.0 |

### Example 15

### Preparation of 50% W/W Picoplatin Suspension in MCT Oil

### Objective

To prepare final concentration of 50% w/w picoplatin suspension in oil. To test microfluidization efficiency with normal saline

TABLE XLII
Materials:

| | |
|---|---|
| Compound | F-80 |

Composition (% w/w)

| | |
|---|---|
| Picoplatin | 10 |
| Miglyol MCT | 80 |
| PL-90 | 10 |
| Normal saline | 10 |
| Total | 110 |

Composition (g/tube)

| | |
|---|---|
| Picoplatin | 3 |
| Miglyol MCT | 24 |
| PL-90 | 3 |
| Normal saline | 3 |
| Total | 33 |

### Procedure:

Weigh out Picoplatin into a 50 mL Falcon tube. Record weight.
Add oil, PL90, and N.S. Record weight.
Mix using a high shear mixer, IKA @ 5 setting for 3 minutes
Microfluidize using Z-chamber for 200 passes. Record final particle size
Let the sample settle down and remove 90% of sample weight of supernatant to obtain 50% w/w suspension.
HPLC for purity
Store at 2-8° C.

### Results:

F80 was able to be microfluidized. The particle size after microfluidization for 200 passes is 554 nm by LLS.

TABLE XLIII
Purity % by HPLC (Method #1) for F80

| Peak Area | RT | Std | |
|---|---|---|---|
| (% of total) | (min) | (0.5 mg/mL in NS) | F-80 |
| Picoplatin | 7.6 | 91.9 | 72.6 |
| Aqua 1 | 4.7 | 3.5 | 12.2 |
| Aqua 2 | 10.3 | 4.4 | 13.1 |
| Unk#1 | 4.3 | 0.2 | 2.0 |
| Total | | 100.0 | 99.9 |

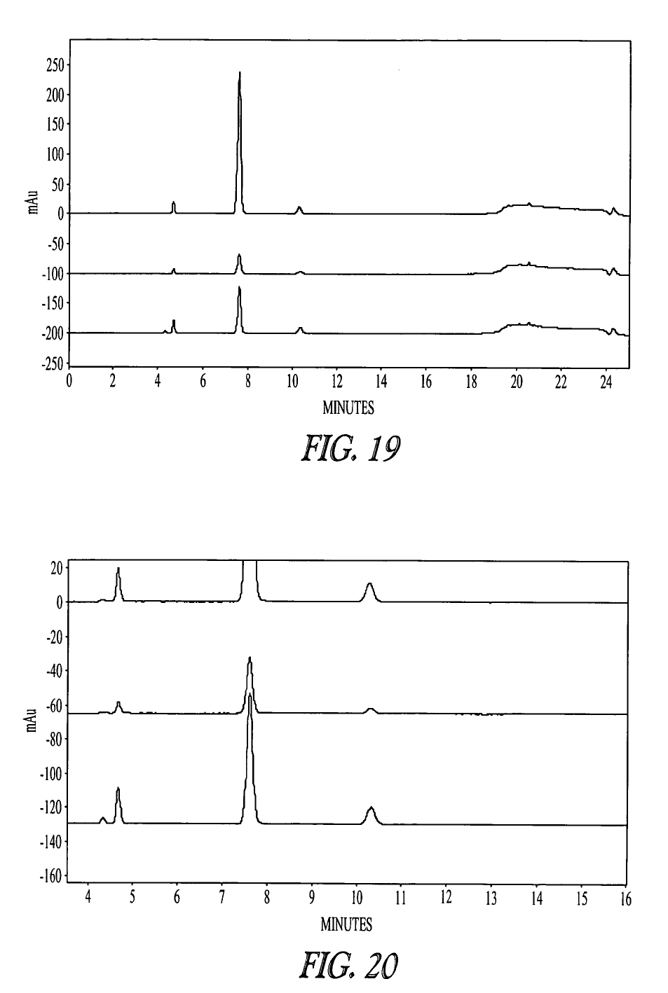

FIG. 19 shows representative HPLC chromatograms. From top down: 0.5 mg/mL standard in normal saline, F77-picoplatin in Ethyl Oleate and PL90, F80-picoplatin in MCT, PL90G and normal saline.

FIG. 20 shows representative HPLC chromatograms, enlarged. From top down: 0.5 mg/mL standard in normal saline, F77-picoplatin in Ethyl Oleate and PL90, F80-picoplatin in MCT, PL90G and normal saline.

### Example 16

### Preparation of 50% W/W Picoplatin Suspension in Ethyl Oleate

### Objective

To prepare final concentration of 50% w/w picoplatin suspension in ethyl oleate at pico:PL90 ratio of 1:1 (wt).

TABLE XLIV
Materials
Composition (% w/w)

| Compound | Supplier Grade | F-81 |
|---|---|---|
| Picoplatin | | 10 |
| Ethyl oleate | | 80 |
| PL-90 | | 10 |
| Total | | 100 |

Composition (g/tube)

| Compound | lot | F-81 |
|---|---|---|
| Picoplatin | | 3 |
| Ethyl oleate | | 24 |
| PL-90 | | 3 |
| Total | | 30 |

### Procedure:

Weigh out Picoplatin into a 50 mL Falcon tube. Record weight. Add oil and PL90. Record weight. Mix using a high shear mixer, IKA @ 5 setting for 3 minutes.

Microfluidize using Z-chamber for 2000 strokes. Record the pass# and final particle size. Let the sample settle down and remove 21 g (90% of sample weight) of supernatant to obtain 50% w/w suspension. HPLC for purity Store at 2-8° C.

### Results:

F81 can be microfluidized. The particle size after microfluidization for 200 passes is 586 nm by LLS.

TABLE XLV
Purity % by HPLC for F81

| Peak Area | RT | Std | |
|---|---|---|---|
| (% of total) | (min) | (0.5 mg/mL in NS) | F-81 |
| Picoplatin | 7.8 | 94.8 | 86.5 |
| Aqua 1 | 4.7 | 2.2 | 6.7 |
| Aqua 2 | 10.3 | 2.9 | 6.6 |
| Unk#1 | 4.3 | 0.0 | 0.3 |
| Unk#2 | 16.5 | 0.1 | 0.0 |
| Total | | 100.0 | 100.01 |

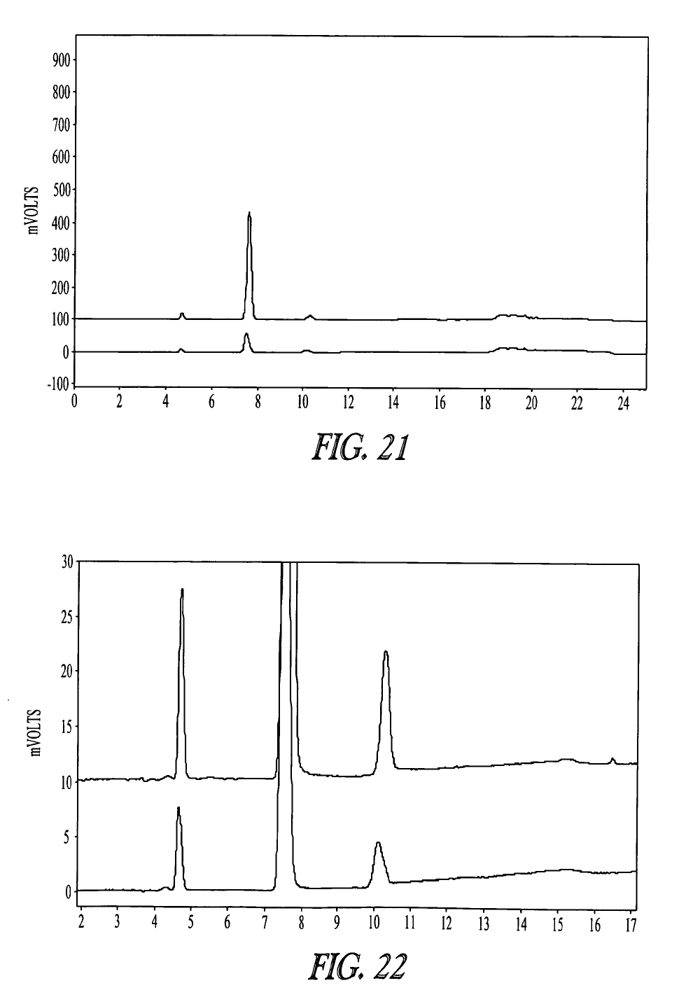

FIG. 21 shows representative HPLC Chromatograms. From top down: 0.5 mg/mL picoplatin standard in normal saline and 0.5 mg/mL F81-picoplatin in PL90 and EO in normal saline.

FIG. 22 shows representative HPLC chromatograms, enlarged. From top down: 0.5 mg/mL picoplatin standard in normal saline and 0.5 mg/mL F81-picoplatin in PL90 and EO in normal saline.

TABLE XLVI
Picoplatin Oil Nano-Suspension Summary

| | PL90:Pico | Pass# | Size (nm) | Observation |
|---|---|---|---|---|
| F74 in MCT | 1:1 | 200 | 482 | Uniformly separated |
| | | | | particles |
| F77 in EO | 1:2 | 200 | 919 | Uniformly separated |
| | | | | particles. |
| F80 in MCT | 1:1 | 200 | 554 | Uniformly separated |
| w/ NS | | | | particles |
| F81 in EO | 1:1 | 200 | 586 | Uniformly separated |
| | | | | particles. |

All publications, patents and patent applications are incorporated herein by reference. While in the foregoing specification this invention has been described in relation to certain preferred embodiments thereof, and many details have been set forth for purposes of illustration, it will be apparent to those skilled in the art that the invention is susceptible to additional embodiments and that certain of the details described herein may be varied considerably without departing from the basic principles of the invention.

1. A formulation for picoplatin adapted for oral administration of the picoplatin, the formulation comprising:
(a) a self-emulsifying formulation containing picoplatin wherein the picoplatin is in a nanoparticulate or microparticulate form,
(b) a plurality of stabilized picoplatin nanoparticles,
(c) a picoplatin solid dispersion in a water-dispersible matrix material, or
(d) a nanoparticulate picoplatin suspension in an oil,
or any combination thereof.

2. The formulation of claim 1, the formulation comprising the self-emulsifying formulation containing picoplatin, wherein the self-emulsifying formulation is prepared by a solvent method; the plurality of picoplatin nanoparticles, wherein the nanoparticles are stabilized with casein or a caseinate and are prepared by microfluidization or high-shear mixing; the picoplatin solid dispersion in a water-dispersible matrix material, wherein the dispersion is prepared by a hot melt method; or the nanoparticulate picoplatin suspension in oil, wherein the oil comprises a medium chain triglyceride or in a fatty ester, or any combination thereof.

3-4. (canceled)

5. The formulation of claim 1 wherein the self-emulsifying formulation comprises an oil, and an emulsifier comprising a lecithin, a surfactant, a PEG, or any combination thereof.

6. The formulation of claim 1 wherein the self-emulsifying formulation comprises at least about 10% w/w, or at least about 5% w/w, of the picoplatin.

7. The self-emulsifying formulation of claim 1 further comprising a first solvent.

8. The formulation of claim 7, wherein the first solvent comprises a dipolar aprotic solvent, a polyethylene glycol, a polyethyleneglycol ether, a polyethyleneglycol derivative of a mono- or di-glyceride, or any combination thereof.

9. (canceled)

10. The formulation of claim 5 wherein the oil comprises a medium chain triglyceride, castor oil, a medium chain mono-glyceride, a medium chain di-glyceride, an edible vegetable oil, peanut oil, cottonseed oil, or soybean oil, or any combination thereof.

11. The formulation of claim 5 wherein the lecithin comprises a high phosphatidyl-choline content lecithin, a low phosphatidylcholine content lecithin, or any combination thereof.

12. The formulation of claim 5 wherein the PEG comprises PEG-400.

13. The formulation of claim 5 wherein the surfactant comprises a mixture composed of about 30% mono-, di-, and triglycerides of C8 and C10 fatty acids, 50% of mono- and di-esters of polyethyleneglycol (PEG 400), and 20% of free PEG 400, or comprises a mixture of glycerol polyethylene glycol oxystearate with fatty acid glycerol polyglycol esters, polyethylene glycols, and glycerol ethoxylate, or comprises a nonionic solubilizer made by reacting castor oil with ethylene oxide in a molar ratio of 1:35, or comprises a PEG-ylated glyceride of lauric acid, or comprises sorbitan mono-9-octadecanoate poly(oxy-1,2-ethanediyl) derivatives, or comprises lecithin, or comprises D-alpha-tocopheryl polyethylene glycol 1000 succinate, or any combination thereof.

14. A method of preparation of the self-emulsifying formulation of claim 1 comprising dissolving picoplatin in a first solvent other than DMSO to provide a picoplatin solution, then, adding an oil and an emulsifier, wherein the emulsifier comprises a lecithin, a PEG, or a surfactant, or any combination thereof; then, adding a second solvent to dissolve picoplatin solution, the oil and the emulsifier, providing a substantially homogeneous second solution; then, evaporating at least the second solvent and, optionally, the first solvent, from the substantially homogeneous second solution to provide the self-emulsifying formulation.

15. The method of claim 14, wherein the first solvent comprises a dipolar aprotic solvent, a polyethylene glycol, a polyethyleneglycol ether, a polyethyleneglycol derivative of a mono- or di-glyceride, or any combination thereof.

16-21. (canceled)

22. The method of claim 14 wherein the picoplatin comprises at least about 10% w/w, or at least about 5% w/w, of the self-emulsifying formulation.

23. A method of treating cancer in a patient in need thereof, comprising administering to the patient the self-emulsifying formulation of claim 5, in a dose, at a frequency, and for a period of time sufficient to provide a beneficial effect to the patient.

24. The formulation of claim 1 comprising a plurality of stabilized picoplatin nanoparticles.

25. The formulation of claim 24 wherein the picoplatin nanoparticles are stabilized with casein, a caseinate, or lecithin, or any combination thereof.

26. (canceled)

27. The formulation of claim 24 comprising at least about 10% w/w of the picoplatin on a dry weight basis.

28. The formulation of claim 24 wherein the picoplatin nanoparticles have an average particle diameter of less than about 1 micron, or less than about 0.5 micron, or less than about 0.25 micron, or less than about 0.15 micron.

29-31. (canceled)

32. A method of preparation of the formulation of claim 24, comprising mixing a stabilizer and an aqueous medium under high-shear conditions or microfluidization conditions, or both, to obtain a uniform dispersion, then adding solid picoplatin and then mixing until an average particle size of the picoplatin is less than about one micron or until crystalline particles are substantially absent, or both, to provide a suspension of the stabilized picoplatin nanoparticles.

33. The method of claim 32 wherein the stabilizer comprises casein or a caseinate, or lecithin.

34-36. (canceled)

37. A method of treating cancer in a patient in need thereof, comprising administering to the patient the formulation comprising stabilized picoplatin nanoparticles of claim 24 in a dose, at a frequency, and for a period of time sufficient to provide a beneficial effect to the patient.

38. The formulation of claim 1 comprising a picoplatin solid dispersion in a water-dispersible matrix material.

39. (canceled)

40. The formulation of claim 38 comprising at least about 10% w/w picoplatin.

41. The formulation of claim 38 wherein the water-dispersible matrix material comprises a PEG-ylated glyceride of stearic acid, or comprises a PEG-ylated glyceride of lauric acid, or comprises a Polyethylene-Polypropylene Glycol copolymer of the formula HO(C₂H₄O)ₐ(C₃H₆O)_{b}(C₂H₄O)ₐH with a weight average molecular weight of about 8400, or comprises sorbitan monostearate, or comprises PEG-8000, or comprises a polyvinylpyrrolidone with a molecular weight of about 90,000, or comprises D-alpha-tocopheryl polyethylene glycol 1000 succinate or comprises glyceryl behenate, or any combination thereof.

42-45. (canceled)

46. A method of preparation of the formulation of claim 38, comprising melting a water-dispersible matrix material at an elevated temperature, then, dispersing solid picoplatin in the melt to provide a dispersed picoplatin composition, then, cooling the composition to provide the picoplatin solid dispersion.

47. The method of claim 46 wherein the step of dispersing the picoplatin in the matrix comprises dissolving the picoplatin in the matrix.

47-51. (canceled)

52. A method of treating cancer in a patient in need thereof, comprising administering to the patient the picoplatin solid dispersion in a water-dispersible matrix material of claim 38 in a dose, at a frequency, and for a period of time sufficient to provide a beneficial effect to the patient.

53. The formulation of claim 1 comprising a nanoparticulate suspension of picoplatin in a medium chain triglyceride or in a fatty ester.

54. The formulation of claim 53 comprising about 20% to about 70% w/w picoplatin.

55. (canceled)

56. The formulation of claim 53 wherein the medium chain triglyceride is a triglyceride of capric acid, caprylic acid, or a combination thereof.

57-61. (canceled)

62. A method of preparation of the formulation of claim 53, comprising combining solid picoplatin and a medium chain triglyceride or a fatty ester, then, under conditions comprising microfluidization by high shear mixing, dispersing the picoplatin in the medium chain triglyceride or fatty ester, wherein the picoplatin comprises about 20% to about 70% w/w of the medium chain triglyceride or fatty ester, to provide the nanoparticulate dispersion.

63. The method of claim 62 comprising further combining a lecithin or a sorbitan mono-9-octadecanoate PEG ether, or both.

64-65. (canceled)

66. A method of treating cancer in a patient in need thereof, comprising administering to the patient the picoplatin solid dispersion in a water-dispersible matrix material of claim 53, in a dose, at a frequency, and for a period of time sufficient to provide a beneficial effect to the patient.

67. The method of claim 23, wherein the cancer is lung cancer, kidney cancer, bladder cancer, renal cancer, stomach and other gastrointestinal (GI) cancers, mesothelioma, melanoma, peritoneal lymphoepithelioma, endometrial cancer, glioblastoma, pancreatic cancer, cervical cancer, testicular cancer, ovarian cancer, colorectal cancer, esophageal cancer, uterine cancer, endometrial cancer, prostate cancer, thymic cancer, breast cancer, head and neck cancer, liver cancer, sarcomas carcinoid tumors, other solid tumors, lymphomas leukemias, or a bone-associated cancer.

68. The method of claim 67 further comprising administration of an effective amount of a second anticancer agent to the patient.

69. The method of claim 68 wherein the second anticancer agent comprises a taxane, a tyrosine kinase and/or a growth factor receptor inhibitor, a cephalotaxine analog, an anti-metabolite, a protein kinase inhibitor, an anthracyclin, a Vinca alkaloid, a podophyllotoxin analog, a growth factor inhibitor, an inhibitor of cell cycle kinases, a cytostatic agent, an alkylating agent, or radiation, or a combination thereof.

70-77. (canceled)
